(19) United States
(12) Patent Application Publication
(10) Pub. No.: US 2025/0269018 A1
(43) Pub. Date: Aug. 28, 2025

(54) COMBINED CANCER THERAPY WITH AN EPITHELIAL CELL ADHESION MOLECULE (EPCAM) INHIBITOR AND A HEPATOCYTE GROWTH FACTOR RECEPTOR (HGFR) INHIBITOR

(71) Applicant: ACADEMIA SINICA, Taipei City (TW)

(72) Inventors: Han-Chung WU, Taipei City (TW); Chi-Chiu LEE, New Taipei City (TW)

(73) Assignee: ACADEMIA SINICA, Taipei City (TW)

(21) Appl. No.: 18/573,623

(22) PCT Filed: Jun. 24, 2022
(86) PCT No.: PCT/US2022/034882

§ 371 Date: Dec. 22, 2023

Related U.S. Application Data
(60) Provisional application No. 63/215,016, filed on Jun. 25, 2021.

(51) Int. Cl. A61K 39/395; A61K 31/4545; A61K 31/47; A61K 31/5377; A61P 35/00

(52) U.S. Cl. CPC A61K 39/39558; A61K 31/4545; A61K 31/47; A61K 31/5377; A61P 35/00

(57) ABSTRACT

The present invention relates to combined therapy of cancer using an epithelial cell adhesion molecule (EpCAM) inhibitor and a hepatocyte growth factor receptor (HGFR) inhibitor. Specifically, the EpCAM inhibitor is an antibody which is directed to an extracellular domain (EpEX) of EpCAM. The combined therapy is effective in inducing apoptosis of cancer cells, inhibiting migration/invasion of cancer cells, reducing tumor size, and/or prolonging survival of a cancer patient.

### RELATED APPLICATIONS

This application claims the benefit of U.S. provisional application No. 63/215,016, filed Jun. 25, 2021 under 35 U.S.C. § 119, the entire content of which is incorporated herein by reference.

### REFERENCE TO SEQUENCE LISTING

This application includes an electronically submitted sequence listing in .txt format. The .txt file contains a sequence listing entitled “5992-0362PUS2_ST25.txt” created on Sep. 3, 2024 and is 11,653 bytes in size. The sequence listing contained in this .txt file is part of the specification and is hereby incorporated by reference herein in its entirety.

### TECHNOLOGY FIELD

The present invention relates to combined therapy of cancer using an epithelial cell adhesion molecule (EpCAM) inhibitor and a hepatocyte growth factor receptor (HGFR) inhibitor. Specifically, the EpCAM inhibitor is an antibody against an extracellular domain (EpEX) of EpCAM. The combined therapy is effective in inducing apoptosis of cancer cells, inhibiting migration/invasion of cancer cells, reducing tumor size, and/or prolonging survival of a cancer patient.

### BACKGROUND OF THE INVENTION

EpCAM is a type I transmembrane protein with 314 amino acids and an observed molecular weight of 39-42 kDa. It contains an extracellular domain (EpEX, 265 amino acids), a single transmembrane domain, and a short intracellular domain (EpICD, 26 amino acids). As a well-known tumor-associated antigen, EpCAM is enriched in various carcinomas, and it is also known to be involved in homotypic cell-cell adhesion in normal epithelium (Dolle et al, 2015). While EpCAM is absent or weakly expressed in the vast majority of healthy epithelial squamous cells, it is strongly expressed in squamous cell carcinomas (Balzar et al, 1999). Furthermore, the expression of EpCAM in squamous carcinomas is correlated with increased cellular proliferation and decreased differentiation (Litvinov et al, 1996). Our group previously developed a neutralizing antibody against EpCAM, EpAb2.6, which has strong potential for use as a colorectal carcinoma (CRC) treatment (Chen et al, 2020; Liang et al, 2018; Liao et al, 2015). Despite its promise as a therapeutic target in CRC, the mechanisms through which EpCAM contributes to tumorigenesis and metastasis are still not completely known.

HGFR (c-MET) is a high affinity receptor tyrosine kinase (RTK) that is activated by hepatocyte growth factor (HGF, also known as Scatter Factor) and encoded by the MET gene (Lai et al, 2009; Peschard & Park, 2007). The tyrosine kinase domain of HGFR contains two tyrosine residues at positions 1234 and 1235, and the phosphorylation of these two sites is essential for the activation of the HGFR receptor (Koch et al, 2020; Ponzetto et al, 1994). Many reports have demonstrated important roles for HGFR in tumorigenesis, cell growth, survival and metastasis (Cao et al, 2019; Li et al, 2018; Mazzone & Comoglio, 2006). In normal tissues, HGFR is expressed in epithelial cells, where it is activated by HGF derived from surrounding mesenchymal cells or in circulation (Birchmeier et al, 2003). Upon HGFR activation by HGF, a morphogenic program is initiated to promote cell migration and invasion. Based on its known functions, HGFR is considered to be a proto-oncogene that normally participates in embryonic development, adult tissue homeostasis and regeneration. Notably, early work on HGFR showed that it has homology with both the growth factor receptor and RTK families (Dean et al, 1985). It was later demonstrated that HGFR is the cognate RTK for HGF, which is identical to the HGFR ligand called scatter factor (Koch et al., 2020; Naldini et al, 1991).

Epithelial-mesenchymal transition (EMT) is associated with cancer progression and metastasis (Iwatsuki et al, 2010). The process of EMT involves a complex series of reversible events that can lead to the loss of epithelial cell adhesion and the induction of a mesenchymal phenotype in cells. Cancer cells that undergo EMT also exhibit enhanced cell motility and invasion through induction of mesenchymal properties and epithelial cell adhesion loss. Indicators of EMT include increased expression of mesenchymal markers, such as Vimentin, Snail and Slug, along with decreased expression of epithelial markers like E-cadherin (Singh & Settleman, 2010). Many reports indicated that HGFR signaling promotes the EMT program, thereby enhancing the invasive and metastatic potential of cancer cells (Gumustekin et al, 2012; Jiao et al, 2016).

EpEX contains two epidermal growth factor (EGF)-like domains, and it may serve as a soluble growth factor in the local tumor microenvironment. A previous report showed that activation of EGF receptor (EGFR) can trigger regulated intramembrane proteolysis (RIP) of EpCAM to induce EMT (Hsu et al, 2016). Of note, EGFR is an RTK that is highly relevant in many types of cancer, since it is overexpressed in a variety of tumors (Normanno et al, 2006). Similar to the effects of EGFR, excessive HGFR activation promotes the growth, survival and migration of cancer cells (Kim et al, 2014; Simiczyjew et al, 2018), acting via a number of downstream effectors, such as AKT, extracellular signal-related kinase (ERK), phosphoinositide 3-kinase, RAS, and SRC (Comoglio et al, 2008; Ortiz-Zapater et al, 2017). Intriguingly, HGFR expression is positively correlated with EGFR expression in basal-type breast cancers (Mueller et al, 2010), and HGFR and EGF family receptors are often co-expressed in cancer cells (Shattuck et al, 2008). Furthermore, EGFR-dependent phosphorylation and activation of HGFR has been reported to occur upon stimulation of epidermal carcinoma cells with EGFR ligands (Jo et al, 2000). Such cross-activation of HGFR in cells with elevated EGFR signaling has also been observed in several types of tumors (Tang et al, 2008). Importantly, however, the mechanisms underlying this cross-activation effect have not been previously identified.

### SUMMARY OF THE INVENTION

Disclosed here is combined use of an epithelial cell adhesion molecule (EpCAM) inhibitor and a HGFR inhibitor for treating cancer. Specifically, the EpCAM inhibitor is an antibody against an extracellular domain (EpEX) of EpCAM. The combined therapy is effective in inducing apoptosis of cancer cells, inhibiting migration/invasion of cancer cells, reducing tumor size, and/or prolonging survival of a cancer patient.

In one aspect, the present invention provides a method for treating cancer, comprising administering to a subject in need thereof
(i) an effective amount of a first inhibitory agent that inhibits the activation of EpCAM signaling; and
(ii) an effective amount of a second inhibitory agent that inhibits the activation of HGFR signaling.

In some embodiments, the first inhibitory agent reduces production (or release) of EpEX, blocks binding of EpEX to HGFR, and/or inhibits EpEX-induced HGFR phosphorylation.

In some embodiments, the second inhibitory agent blocks binding of HGF to HGFR.

In some embodiments, the first inhibitory agent is an antibody directed to EpEX or an antigen-binding fragment thereof.

In some embodiments, the anti-EpEX antibody as described herein specifically binds to epidermal growth factor (EGF)-like domains I and II. In certain examples, the anti-EpEX antibody as described herein has a specific binding affinity to an epitope within the sequence of CVCENYKLAVN (aa 27 to 37) (SEQ ID NO: 20) located in the EGF-like domain I, and KPEGALQNNDGLYDPDCD (aa 83 to 100) (SEQ ID NO: 19) located in the EGF-like domain II.

In some embodiments, the antibody or antigen-binding fragment comprises
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13.

In some embodiments, the VH comprises the amino acid sequence of SEQ ID NO: 15, and/or the VL comprises the amino acid sequence of SEQ ID NO: 16.

In some embodiments, the first inhibitory agent is effective in inhibiting phosphorylation of TACE and PS2 signaling.

In some embodiments, the second inhibitory agent is selected from the group consisting of foretinib, crizotinib and cabozantinib.

In some embodiments, the method of the present invention is effective in inducing apoptosis of cancer cells.

In some embodiments, the method of the present invention is effective in inhibiting migration/invasion of cancer cells and/or reducing tumor size.

In some embodiments, the method of the present invention is effective in prolonging survival of the subject.

In some embodiments, the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer.

In another aspect, the present invention provides a kit of a pharmaceutical composition comprising
(i) an effective amount of a first inhibitory agent that inhibits the activation of EpCAM signaling; and
(ii) an effective amount of a second inhibitory agent that inhibits the activation of HGFR signaling.

Also provided in the present invention is use of a combination of (i) a first inhibitory agent that inhibits the activation of EpCAM signaling; and (ii) (ii) a second inhibitory agent that inhibits the activation of HGFR signaling for manufacturing a medicament or kit for treating cancer.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1A to 1J. EpEX interacts with HGFR and induces HGFR phosphorylation. (FIG. 1A) Immunoprecipitation (IP) of endogenous EpCAM bound to HGFR in HCT116 and HT29 cells. (FIG. 1B) HEK293T cells were transfected with HGFR_{ECD}-c-Myc and EpCAM-V5. IP was performed with control IgG, anti-V5 antibody or anti-c-Myc antibody followed by Western blotting. (FIG. 1C) EpEX-Fc and HGFR-His recombinant protein (2.5 μg/ml) interaction was examined by IP with Dynabeads Protein G and Western blotting with anti-6× His tag antibody. (FIG. 1D) Starved HCT116 and HT29 cells were treated with different doses of EpEX-His for 15 min, and starved HCT116 and HT29 cells were treated with 50 nM EpEX-His for the indicated times. The phosphorylation of HGFR was examined by Western blotting. (FIG. 1E) Wild-type (WT) or EpCAM knockout (KO) HCT116 and H29 cells were starved for 16 h then treated with EpEX-His (50 nM) for 15 min. The level of phosphorylated HGFR was assayed with an ELISA kit (ab126451). (FIG. 1F) HEK293T cells were transfected with HGFR_{ECD}-c-Myc and full length or EGF like-domain deletion mutant EpCAM-V5. The protein interaction was probed by IP with anti-V5 or anti-c-Myc antibody and Western blotting with anti-V5 or anti-cMyc antibody. (FIG. 1G) HEK293T cells were transfected with c-Myc or HGFR_{ECD}-c-Myc and full length or EGF like-domain deletion mutant EpEX-His. The protein interaction was probed by IP with anti-c-Myc antibody and Western blotting with anti-cMyc and anti-His antibody. (FIG. 1H) HGFR-His recombinant protein (2 μg/ml) was added to EGF like-domain deletion mutant-Fc-coated (1 μg/ml) ELISA plates and detected by TMB colorimetric peroxidase assay. HCT116 cells were starved and treated with wild-type or EGF-domain deletion mutant EpEX, and phosphorylated HGFR was analyzed by (FIG. 1I) Western blotting and (FIG. 1J) ELISA kit (ab126451). All data are presented as mean±SEM. *, p<0.05.

FIGS. 2A to 2K. EpEX promotes tumor progression via HGFR signaling. (FIG. 2A) WT or EpCAM knockout (KO) HCT116 and HT29 cells treated with HGF (0.5 nM) for 15 min. The phosphorylation of HGFR, AKT, and ERK was examined by Western blotting. (FIG. 2B) WT or KO HCT116 and HT29 cells were treated with HGF (0.5 nM) with 2% FBS for the indicated times. Cell growth was examined by the WST-1 assay. (FIG. 2C) Starved HCT116 cells were treated with HGFR inhibitor SU11274 (SU, 10 μM) for 1 h then treated with 50 nM of EpEX-His for 15 min. The levels of phosphorylated HGFR, EGFR, AKT, and ERK were examined by Western blotting. HCT116 and HT29 cells were treated with 50 nM EpEX and SU (10 μM). (FIG. 2D) Cell growth was examined by WST-1 assay after treatment for indicated time. (FIG. 2E) HCT116 cells were treated with shLuc or HGFR shRNA then treated with 50 nM of EpEX-His for 15 min. The levels of phosphorylated HGFR, EGFR, ERK, and AKT were examined in HGFR knockdown HCT116 cells by Western blotting. (FIG. 2F) The levels of phosphorylated ADAM17 and presenilin 2 were examined by Western blotting. (FIG. 2G) Active β-catenin nuclear translocation was assay by using Western blotting. (H) HCT116 cells were treated with shLuc or HGFR shRNA then treated with 50 nM of EpEX-His for indicated times. Cell growth was examined by the WST-1 assay. (FIG. 2I) HCT116 cells after shLuc and shHGFR treatment were treated with 50 nM of EpEX-His with 2% FBS for 7 days. Colony formation was examined by crystal violet staining. Quantification of the nuclear (3-catenin in the bottom panel. (FIG. 2J) HCT116 cells were treated with HGF (0.5 nM) for indicated times, and EpEX protein level in culture medium was examined by immunoprecipitation and Western blotting. (FIG. 2K) HCT116 cells were treated with HGF (0.5 nM) for 15 min. The phosphorylated HGFR, presenilin 2 and ADAM17 protein level in cell lysates was analyzed by Western blotting and EpEX protein level in culture medium was examined by immunoprecipitation and Western blotting. All data are presented as mean±SEM. *, p<0.05; **, p<0.01.

FIGS. 3A to 3H. EpEX induces the activation of ERK and FAK-AKT signaling pathway. Wild type (WT) or EpCAM knockout (KO) HCT116 cells after EpEX-His treatment. (FIG. 3A) The levels of phosphorylated HGFR, AKT, FAK, GSK3β, ERK, ADAM17, and presenilin 2 were assayed by Western blotting. (FIG. 3B) Colony formation was examined by crystal violet staining. (FIG. 3C) HCT116 cells were treated with TAPI (ADAM17 inhibitor) or DAPT (γ-secretase inhibitor) for 24 hours, and the phosphorylation of HGFR, AKT, and ERK was analyzed by Western blotting, and EpEX protein level in culture medium was examined by immunoprecipitation and Western blotting. (FIG. 3D) HCT116 and HT29 cells were starved for 16 h, then treated with EpEX-His (50 nM) and HGF (0.5 nM) for 15 min. The levels of phosphorylated HGFR, AKT, and ERK were examined by Western blotting. (FIG. 3E) HCT116 cells were starved for 16 h, then treated with 50 nM EpEX-His for 15 min. HGFR inhibitor SU11274 (SU, 10 μM), AKT inhibitor LY294002 (LY, 25 μM), ERK inhibitor U0126 (U0, 20 μM), or FAK inhibitor PF-562271 (PF, 10 μM) were applied 1 h before EpEX treatment. Phosphorylation of AKT, ERK, and FAK was examined by Western blotting. (FIG. 3F) HCT116 cells were starved for 16 h, then treated with 50 nM EpEX, and SU (10 μM), LY (25 μM), U0 (20 μM) or PF (10 μM). Colony formation was examined by crystal violet staining after treatment for 7 days. The relative colony densities are shown. Migration ability was examined by the (FIG. 3G) wound healing assay at the indicated times. (FIG. 3H) The numbers of migration cell was assessed by a Transwell after treatment for 24 h. All data are presented as mean±SEM. *, p<0.05.

FIGS. 4A to 4L. EpEX induces EMT and invasion by stabilizing β-catenin and Snail through decreasing GSK3β activity. (FIG. 4A) The protein expression of EMT markers and regulators was detected by Western blotting in Wild-type (WT) or EpCAM knockout (KO) HCT116 cells after EpEX treatment. (FIG. 4B) Cell invasion was examined by Transwell chamber assay with matrigel. (FIG. 4C) WT or KO HCT116 and HT29 cells were starved for 16 h, then treated with 0.5 nM of HGF with 2% FBS for 24 h. EMT-related protein expression (E-cadherin, vimentin, and Snail) was examined by Western blotting. (FIG. 4D) WT or KO HCT116 and HT29 cells were treated with HGF (0.5 nM) for 24 h. Invasion by HCT116 and HT29 cells was examined by Transwell chamber assay with matrigel. (FIG. 4E) HCT116 and HT29 cells were starved for 16 h, then treated with 0.5 nM of HGF with 2% FBS for 24 h. EMT-related protein expression (E-cadherin, Vimentin, and Snail) was examined by Western blotting. (FIG. 4F) HCT116 and HT29 cells were starved for 16 h, then treated with 0.5 nM of HGF with 2% FBS for 24 h. Cell invasion was assessed by a Transwell assay with matrigel. (FIG. 4G) HCT116 cells after shLuc and shHGFR treatment were treated with 50 nM of EpEX-His. HCT116 cells after shLuc and shHGFR treatment were treated with 50 nM of EpEX-His with 2% FBS for 24 h. EMT-related protein expression (E-cadherin, Vimentin, and Snail) was examined by Western blotting. (FIG. 4H) Cell invasion was examined by Transwell chamber assay with matrigel. (FIG. 4I) Starved HCT116 cells were treated SU (10 μM) for 1 h, followed by treatment with EpEX-His (50 nM) for 24 h. Phosphorylated GSK3β, active-β-catenin, and Snail were detected by Western blotting. (FIG. 4J) HCT116 cells were starved for 16 h, then treated with 50 nM EpEX-His for 24 min. AKT inhibitor LY294002 (25 μM), ERK inhibitor U0126 (20 μM), or PF-562271 (10 μM) were applied 1 h before EpEX treatment. Protein expression of phosphorylated GSK3β and Snail was examined by Western blotting. (FIG. 4K) HCT116 cells were starved for 16 h, then treated with 50 nM EpEX, and SU (10 μM), LY (25 μM), UO (20 μM) or PF (10 μM). Cell invasion was examined by Transwell chamber assay with matrigel. (FIG. 4L) Protein expression was analyzed by Western blotting in EpEX-His (50 nM) treated HCT116 cells after treatment with or without 2 μM GSK3β inhibitor (BIO) for 24 h. Quantification of the normalized protein expression in the right panel. All data are presented as mean±SEM. *, p<0.05; **,p<0.01.

FIGS. 5A to 5I. EpEX promotes Snail protein stability through inhibition of ubiquitination-mediated proteasomal degradation. (FIG. 5A) The gene expression of EMT markers and regulators was detected by qRT-PCR in Wild-type (WT) or EpCAM knockout (KO) of HCT116 and HT29 cells. (FIG. 5B) Stability of Snail protein in WT or KO of HCT116 cells. Cells were treated with cyclohexamide (CHX) 100 μg/ml at the indicated intervals and subjected to Western blotting. (FIG. 5C) The protein expression of Snail was analyzed in WT or KO HCT116 cells by treating with or without 10 mM MG132 (proteasome inhibitor) for 6 h, followed by Western blotting. (FIG. 5D) WT or KO HCT116 cells were treated with 10 μM MG132 for 6 h before cell collection. The lysates were subjected to immunoprecipitation using anti-Snail antibody and input. Western blotting was performed with the indicated antibodies to detect ubiquitinated Snail protein. (FIG. 5E) Stability of Snail protein in HCT116 cells after EpEX (50 nM) treatment for 24 h. Cells were treated with cyclohexamide (CHX; 100 μg/ml) for the indicated intervals and then subjected to Western blotting. (FIG. 5F) Expression of the gene encoding SNAIL was detected by qRT-PCR in HCT116 cells after EpEX (50 nM) treatment for 24 h. (FIG. 5G) The protein expression of Snail in HCT116 was analyzed cells by Western blotting after EpEX (50 nM) treatment for 24 h, and treatment with or without 10 μM MG132 (proteasome inhibitor) for 6 h. (FIG. 5H) Schematic representation of positions of mutant within Snail phosphorylation motifs. (FIG. 5I) HCT116 cells were transfected with Snail-WT, 2SA, 4SA, and 6SA for 24 h and then further treated with or without EpEX (50 nM) for 24 h. The expression of Snail was detected by Western blotting. All data are presented as mean±SEM. *, p<0.05; **, p<0.01.

FIGS. 6A to 6J. EpAb2-6 inhibits EpCAM and HGFR signaling and promotes active β-catenin and Snail protein degradation via activating GSK3β. (FIG. 6A) HCT116 cells were treated with 10 μg/ml control IgG (normal mouse IgG, NMIgG) or mouse EpAb2-6 (EpAb2-6) for 16 h, followed by treatment with EpEX-His (50 nM) for 15 min. Levels of phosphorylated HGFR, AKT, FAK, GSK3β, ERK, ADAM17, and presenilin 2 were examined by Western blotting. (FIG. 6B) HCT116 cells were treated with 10 μg/ml control IgG or mouse EpAb2-6 for 16 h, followed by treatment without or with HGF (0.5 nM) for 15 min. Levels of phosphorylated HGFR, AKT, and ERK were examined by Western blotting. HCT116 cells were treated with mouse EpAb2-6 (10 μg/ml) and HGF (0.5 nM). (FIG. 6C) Cell migration was examined by the wound healing assay at the indicated times. (FIG. 6D) Cell invasion was assessed by a Transwell assay with matrigel after 24 h. (FIG. 6E) HCT116 cells were treated with NMIgG or EpAb2-6 for 6 h and then immunoprecipitated with anti-EpCAM (IP: EpCAM) or anti-HGFR (IP: HGFR) antibodies, followed by Western blotting. (FIG. 6F) EpEX-His (2 μg/ml) co-treated with 1 μg IgG or EpAb2-6 was added to HGFR-Fc-coated (1 μg/ml) ELISA plates and detected by TMB colorimetric peroxidase assay. (FIG. 6G) EMT associated protein levels were detected by Western blotting in HCT116 cells treated with NMIgG or EpAb2-6 for 24 h. (FIG. 6H) Protein expression was analyzed by Western blotting in HCT116 cells after treatment with EpAb2-6 and 2 μM GSK3β inhibitor (BIO) for 24 h. Quantification of the normalized protein expression in the bottom panel. (FIG. 6I) HCT116 cells were treated with 10 μM MG132 and EpAb2-6 for 6 h before cell collection and subsequent Western blotting. (FIG. 6J) Stability of Snail protein in HCT116 cells treated with NMIgG or EpAb2-6. Cells were treated with cyclohexamide (CHX) 100 μg/ml at the indicated intervals and subjected to Western blotting. Bottom graph shows quantification of Snail half-life in indicated groups. All data are presented as mean±SEM. *, p<0.05; **, p<0.01.

FIGS. 7A to 7G. EpAb2-6 binds EpEX and induces apoptosis by F(ab′)₂ and inhibits regulated intramembrane proteolysis (RIP) activation and HGFR signaling. (FIG. 7A) Binding affinity of IgG EpAb2-6 (mouse) and F(ab′)2 to EpEX-His (1 μg/ml) coated overnight was checked using ELISA (OD450). (FIG. 7B) HCT116 cells were treated with 100 μg/ml control IgG, Fc, or F(ab′)₂ of EpAb2-6 for 24 h. The apoptotic and necrotic cells were quantified by fluorescein annexin V-FITC/PI double labeling. (FIG. 7C) HCT116 and HT29 cells were treated with 10 μg/ml control IgG, MT201, humanized EpAb2-6 (hEpAb2-6) or mouse hybridoma EbAb2-6 (mEpAb2-6) for 24 h. The apoptotic and necrotic cells were quantified by fluorescein annexin V-FITC/PI double labeling. (FIG. 7D) HCT116 cells were treated with 10 μg/ml control IgG, MT201, hEpAb2-6 or mEpAb2-6 for 16 h, followed by treatment with EpEX-His (50 nM) for 15 min. Levels of phosphorylated HGFR, AKT, and ERK, and (FIG. 7E) RIP proteins ADAM17 and presenilin 2 were examined by Western blotting. Anti-EpCAM antibody and crizotinib coordinately induces apoptosis in colon cancer cells. (FIG. 7F) HCT116 and HT29 cells were treated with 10 μg/ml NMIgG or EpAb2-6 and 4 μM HGFR inhibitor crizotinib for 24 h. The apoptotic and necrotic cells were quantified by fluorescein annexin V-FITC/PI double labeling. (FIG. 7G) HCT116 and HT29 cells were treated with 10 μg/ml NMIgG or EpAb2-6 and 10 μM HGFR inhibitor crizotinib. Cell invasion was assessed by a Transwell assay with matrigel after 24 h. All data are presented as mean±SEM. *, p<0.05.

FIGS. 8A to 8G. EpAb2-6 binds to both EGF-like domain I and II of EpCAM. HEK293T cells were transfected with full length or EGF like-domain deletion mutant EpCAM-V5. Antibody binding was assessed by (FIG. 8A) Western blotting, (FIG. 8B) flow cytometry, and (FIG. 8C) immunofluorescence. (FIG. 8D) EpCAM mutants were constructed with amino acid substitutions in the EGF-I (Y32A) and EGF-II (L94A, Y95A, or D96A) domains. EpCAM wild-type and mutant proteins were expressed in HEK293T cells. Binding of MT201, EpAb2-6 and EpAb23-1 to EpCAM wild-type and mutants were evaluated by (FIG. 8E) immunofluorescence, (FIG. 8F) flow cytometry, and (FIG. 8G) cellular ELISA. All data are presented as mean±SEM. *, p<0.05; **,p<0.01.

FIGS. 9A to 9K. EpAb2-6 and crizotinib coordinately inhibit tumor progression and metastasis. (FIG. 9A) Timeline of the experiment to evaluate EpAb2-6 and/or crizotinib effects in the in a metastatic animal model. (FIG. 9B) NOD/SCID mice were intravenously injected with 5×10⁶HCT116 cells, followed by treatment with either control IgG, EpAb2-6 and/or crizotinib (n=5). The survival curve, median survival days and representative H&E staining of lung tissues in metastatic animal models. (FIG. 9C) Timeline of the experiment to evaluate EpAb2-6 and/or crizotinib in the in orthotopic animal models. (FIG. 9D) NOD/SCID mice received orthotopic implantation of HCT116-Luc cells and then were treated with control IgG (normal mouse IgG, NMIgG), crizotinib, EpAb2-6, or crizotinib combined with EpAb2-6 starting at 3 days after tumor inoculation (n=5). Tumor growth was monitored by examining bioluminescence with the IVIS 200 Imaging System. (FIG. 9E) HCT116-Luc tumor cells monitored by bioluminescence quantification. (FIG. 9F) Bodyweights of each treatment group in HCT116 orthotopic animal models after indicated treatments. (FIG. 9G) Survival curves and median survival days of each treatment group in HCT116 orthotopic animal models. (FIG. 9H) NOD/SCID mice were orthotopically implanted with HT29-Luc cells and then treated with control IgG, crizotinib, EpAb2-6, or crizotinib combined with EpAb2-6 starting at 3 days after tumor inoculation (n=5). Tumor growth was monitored by examining bioluminescence with an IVIS 200 Imaging System. (FIG. 9I) HT29-Luc tumor cells monitored by bioluminescence quantification. (FIG. 9J) Mice bodyweight of each treatment group in HT29 orthotopic animal models after indicated treatments. (FIG. 9K) Survival curves and median survival days of each treatment group in HT29 orthotopic animal models. All data are presented as mean±SEM. *, p<0.05; **, p<0.01.

FIGS. 10A to 10B. Sequence features and domains of human EpCAM. (FIG. 10A) Full length of human EpCAM containing 314 amino acid residues (SEQ ID NO: 17). (FIG. 10B) identification of domains of EpCAM where the EpEX domain includes EGF I domain (aa 27-59) covering VGAQNTVIC (aa 51 to 59, SEQ ID NO: 18) and EGF II domain (aa 66-135) covering KPEGALQNNDGLYDPDCDE (aa 83 to 100, SEQ ID NO: 19) with the LYD motif (aa 94-96).

FIG. 11. The amino acid sequences of EpAb2-6, in which a VH (SEQ ID NO: 15) comprising HC CDR1 of SEQ ID NO: 2, HC CDR2 of SEQ ID NO: 4, and HC CDR3 of SEQ ID NO: 6; and a VL (SEQ ID NO: 16) comprising LC CDR1 of SEQ ID NO: 9, LC CDR2 of SEQ ID NO: 11, and HC CDR3 of SEQ ID NO: 13.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, 20 or 10 amino acid residues).

As used herein, the term “approximately” or “about” refers to a degree of acceptable deviation that will be understood by persons of ordinary skill in the art, which may vary to some extent depending on the context in which it is used.

Specifically, “approximately” or “about” may mean a numeric value having a range of ±10% or +5% or +3% around the cited value.

As used herein, the term “substantially identical” refers to two sequences having 80% or more, preferably 85% or more, more preferably 90% or more, even more preferably 95% or more homology.

As used herein, the term “antibody” (interchangeably used in plural form, antibodies) means an immunoglobulin molecule having the ability to specifically bind to a particular target antigenic molecule. As used herein, the term “antibody” includes not only intact (i.e. full-length) antibody molecules but also antigen-binding fragments thereof retaining antigen binding ability e.g. Fab, Fab′, F(ab′)2 and Fv. Such fragments are also well known in the art and are regularly employed both in vitro and in vivo. The term “antibody” also includes chimeric antibodies, humanized antibodies, human antibodies, diabodies, linear antibodies, single chain antibodies, multispecific antibodies (e.g., bispecific antibodies) and any other modified configuration of the immunoglobulin molecule that comprises an antigen recognition site of the required specificity, including amino acid sequence variants of antibodies, glycosylation variants of antibodies, and covalently modified antibodies.

An intact or complete antibody comprises two heavy chains and two light chains. Each heavy chain contains a variable region (V_{H}) and a first, second and third constant regions (C_{H}1, C_{H}2 and C_{H}3); and each light chain contains a variable region (V_{L}) and a constant region (C_{L}). The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light chains and those of heavy chains are responsible for antigen binding. The variables regions in both chains are responsible for antigen binding generally, each of which contain three highly variable regions, called the complementarity determining regions (CDRs); namely, heavy (H) chain CDRs including HC CDR1, HC CDR2, HC CDR3 and light (L) chain CDRs including LC CDR1, LC CDR2, and LC CDR3. The three CDRs are franked by framework regions (FR1, FR2, FR3, and FR4), which are more highly conserved than the CDRs and form a scaffold to support the hypervariable regions. The constant regions of the heavy and light chains are not responsible for antigen binding, but involved in various effector functions. Depending on the antibody amino acid sequence of the constant domain of its heavy chains, immunoglobulins can be assigned to different classes. There are five major classes of immunoglobulins: IgA, IgD, IgE, IgG, and IgM. The heavy-chain constant domains that correspond to the different classes of immunoglobulins are called alpha, delta, epsilon, gamma, and mu, respectively.

As used herein, the term “antigen-binding fragment” or “antigen-binding domain” refers to a portion or region of an intact antibody molecule that is responsible for antigen binding. An antigen-binding fragment is capable of binding to the same antigen to which the parent antibody binds. Examples of antigen-binding fragments include, but are not limited to: (i) a Fab fragment, which can be a monovalent fragment composed of a V_{H}-C_{H}1 chain and a V_{L}-C_{L} chain; (ii) a F(ab′)2 fragment which can be a bivalent fragment composed of two Fab fragments linked by a disulfide bridge at the hinge region; (iii) a Fv fragment, composed of the V_{H} and V_{L} domains of an antibody molecule associated together by noncovalent interaction; (iv) a single chain Fv (scFv), which can be a single polypeptide chain composed of a V_{H} domain and a V_{L} domain via a peptide linker; and (v) a (scFv)₂, which can contain two V_{H} domains linked by a peptide linker and two V_{L} domains, which are associated with the two V_{H} domains via disulfide bridges.

As used herein, the term “chimeric antibody” refers to an antibody containing polypeptides from different sources, e.g., different species. In some embodiments, in chimeric antibodies, the variable region of both light and heavy chains may mimic the variable region of antibodies derived from one species of mammal (e.g., a non-human mammal such as mouse, rabbit and rat), while the constant region may be homologous to the sequences in antibodies derived from another mammal such as a human.

As used herein, the term “humanized antibody” refers to an antibody comprising a framework region originated from a human antibody and one or more CDRs from a non-human (usually a mouse or rat) immunoglobulin.

As used herein, the term “human antibody” refers to an antibody in which essentially the entire sequences of the light chain and heavy chain sequences, including the complementary determining regions (CDRs), are from human genes. In some circumstances, the human antibodies may include one or more amino acid residues not encoded by human germline immunoglobulin sequences e.g. by mutations in one or more of the CDRs, or in one or more of the FRs, such as to, for example, decrease possible immunogenicity, increase affinity, and eliminate cysteines that might cause undesirable folding, etc.

As used herein, the term “specific binds” or “specifically binding” refers to a non-random binding reaction between two molecules, such as the binding of the antibody to an epitope of its target antigen. An antibody that “specifically binds” to a target antigen or an epitope is a term well understood in the art, and methods to determine such specific binding are also well known in the art. An antibody “specifically binds” to a target antigen if it binds with greater affinity/avidity, more readily, and/or greater duration than it binds to other substances. In other words, it is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, the affinity of the binding can be defined in terms of a dissociation constant (K_{D}). Typically, specifically binding when used with respect to an antibody can refer to an antibody that specifically binds to (recognize) its target with an KD value less than about 10^{˜7} M, such as about 10^{˜8} M or less, such as about 10^{˜9} M or less, about 10^{˜10} M or less, about 10^{˜n} M or less, about 10″¹² M or less, or even less, and binds to the specific target with an affinity corresponding to a K_{D} that is at least ten-fold lower than its affinity for binding to a non-specific antigen (such as BSA or casein), such as at least 100 fold lower, e.g. at least 1,000 fold lower or at least 10,000 fold lower.

As used herein, the term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

As used herein, the term “complementary” refers to the topological compatibility or matching together of interacting surfaces of two polynucleotides. A first polynucleotide is complementary to a second polynucleotide when the nucleotide sequence of the first polynucleotide is identical to the nucleotide sequence of the polynucleotide binding partner of the second polynucleotide. Thus, the polynucleotide whose sequence 5′-ATATC-3′ is complementary to a polynucleotide whose sequence is 5′-GATAT-3′.”

As used herein, the term “encoding” refers to the natural property of specific sequences of nucleotides in a polynucleotide (e.g., a gene, a cDNA, or an mRNA) to serve as templates for synthesis of other polymers and macromolecules in biological processes having either a given sequence of RNA transcripts (i.e., rRNA, tRNA and mRNA) or a given sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” encompasses all nucleotide sequences that are degenerate versions of each other and that encode the same amino acid sequence.

As used herein, the term “recombinant nucleic acid” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence (expression vector) or maintaining the given nucleotide sequence for replicating it, manipulating it or transferring it between different locations (e.g., between different organisms). Vectors can be introduced into a suitable host cell for the above-described purposes. A “recombinant cell” refers to a host cell that has had introduced into it a recombinant nucleic acid. “A transformed cell” mean a cell into which has been introduced, by means of recombinant DNA techniques, a DNA molecule encoding a protein of interest.

Vectors may be of various types, including plasmids, cosmids, episomes, fosmids, artificial chromosomes, phages, viral vectors, etc. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprise, for example and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, a secretion signal sequence (e.g., α-mating factor signal), a stop codon, and other control sequence (e.g., Shine-Dalgarno sequences and termination sequences). Preferably, vectors may further contain a marker sequence (e.g., an antibiotic resistant marker sequence) for the subsequent screening/selection procedure. For purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes a tag for purpose of purification e.g. a His-tag.

As used herein, the term “treatment” refers to the application or administration of one or more active agents to a subject afflicted with a disorder, a symptom or condition of the disorder, or a progression of the disorder, with the purpose to cure, heal, alleviate, relieve, alter, remedy, ameliorate, improve, or affect the disorder, the symptom or condition of the disorder, the disabilities induced by the disorder, or the progression or predisposition of the disorder.

The present disclosure is based, at least in part, on the development of combined cancer therapy using an epithelial cell adhesion molecule (EpCAM) inhibitor and an HGFR inhibitor.

EpEX, an extracellular domain of EpCAM, contains two epidermal growth factor (EGF)-like domains, and it may serve as a soluble growth factor in the local tumor microenvironment. Activation of EGF receptor (EGFR) can trigger regulated intramembrane proteolysis (RIP) of EpCAM to induce epithelial-mesenchymal transition (EMT) (Hsu et al., 2016). EGFR is an RTK that is highly relevant in many types of cancer, since it is overexpressed in a variety of tumors (Normanno et al, 2006). Excessive HGFR activation promotes the growth, survival, and migration of cancer cells (Normanno et al, 2006) via a number of downstream effectors, such as AKT, extracellular signal-related kinase (ERK), phosphoinositide 3-kinase, RAS, and SRC (Comoglio et al, 2008; Ortiz-Zapater et al, 2017). HGFR expression is positively correlated with EGFR expression in basal-type breast cancers (Comoglio et al, 2008; Ortiz-Zapater et al, 2017), and HGFR and EGF family receptors are often co-expressed in cancer cells (Comoglio et al, 2008; Ortiz-Zapater et al, 2017). Furthermore, EGFR-dependent phosphorylation and activation of HGFR occur upon stimulation of epidermal carcinoma cells with EGFR ligands (Comoglio et al, 2008; Ortiz-Zapater et al, 2017). Such cross-activation of HGFR in cells with elevated EGFR signaling are observed in several tumor types as well (Tang et al, 2008).

In the present invention, it is surprisingly found that EpEX binds to HGFR and activates its downstream signaling to promote cell proliferation, migration and invasion. It is also found that EpCAM neutralizing antibody, EpAb2-6, attenuates phosphorylation of HGFR and inhibits cancer cell metastasis. Thus, the results of this study provide a mechanistic rationale for simultaneous targeting of EpCAM and HGFR signaling to combat cancer metastasis.

As used herein, “combined therapy” refers to treatment that combines two or more therapeutic agents or approaches. “Combination” means that two or more therapeutic agents or approaches are given to the same subject, at the same time or in sequence. Preferably, combined therapy provides synergistic effects.

As used herein, the term “synergistic effect” may mean and include a cooperative action resulted in a combination of two or more active agents in which the combined activity of the two or more active agents exceeds the sum of the activity of each active agent alone. The term “synergistic effect” may also refer to that two or more active agents when used together provide combined activity such that a lower dose of each may be used to achieve comparative or enhanced activity when single agent is used.

Therefore, the present invention provides a combined therapy for treating cancer, comprising administering to a subject in need thereof a combination comprising (i) an effective amount of a first inhibitory agent that inhibits the activation of EpCAM signaling (an EpCAM inhibitor); and (ii) an effective amount of a second inhibitory agent that inhibits the activation of HGFR signaling (an HGFR inhibitor).

In some embodiments, an anti-EpEX antibody as used herein specifically binds to the EGF-like domain I of EpCAM (aa 27-59 of EpCAM) and the EGF-like domain II of EpCAM (aa 66-135 of EpCAM). Specifically, an anti-EpEX antibody as used herein has a specific binding affinity to an epitope within the sequence of CVCENYKLAVN (aa 27 to 37) (SEQ ID NO: 20) located in the EGF-like domain I, and KPEGALQNNDGLYDPDCD (aa 83 to 100) (SEQ ID NO: 19) located in the EGF-like domain II. More specifically, an anti-EpEX antibody as used herein recognizes the NYK motif (aa 31-33) within domain I and the LYD motif (aa 94-96) within domain II in EpCAM. In contrast, a number of other antibodies (e.g. MT201, M97, 323/A3 and edrecolomab) target only the well-described EGF I domain of EpCAM. The distinct features of the anti-EpEX antibody according to the present invention from other antibodies are described below.

| | |
|---|---|
| anti-EpEX antibody | binding to both domain I and domain II and |
| according to the | effective in inducing apoptosis of cancer |
| present invention | cells |
| Other antibodies | binding to domain I only and failing to |
| (e.g. MT201, M97, 323/A3 | induce apoptosis of cancer cells |
| and edrecolomab) | |

One certain anti EpEX antibody as used herein is EpAb2-6 as shown in Examples below. The amino acid sequences of the heavy chain variable region (V_{H}) and light chain variable region (V_{L}), and their complementary determining regions (HC CDR1, HC CDR2 and HC CDR3) (LC CDR1, LC CDR2 and LC CDR3) of EpAb2-6 are as shown in Table 1 below. The anti-EpEX antibody of the present invention includes EpAb2-6 and its functional variant.

TABLE 1
V_{H} domain

| FW1 | CDR1 | FW2 | CDR2 |
|---|---|---|---|
| VKLQESGPELKKPGETVK | GYTFTDYSMH | WVKQAPGKGLKWMG | INTETGEP |
| ISCKAS | (SEQ ID NO: 2) | W | (SEQ ID NO: 4) |
| (SEQ ID NO: 1) | | (SEQ ID NO: 3) | |
| FW3 | CDR3 | FW4 | |
| TYADDFKGRFAFSLETSA | TAVY | WGQGTTVTVSS | |
| STAYLQINNLKNEDTATY | (SEQ ID NO: 6) | (SEQ ID NO: 7) | |
| FCAR | | | |
| (SEQ ID NO: 5) | | | |

V_{L} domain

| FW1 | CDR1 | FW2 | CDR2 |
|---|---|---|---|
| DIQMTQSPSSLSASLGERV | RASQEISVSLS | WLQQEPDGTIKRLIY | ATSTLDS |
| SLTC | (SEQ ID NO: 9) | (SEQ ID NO: 10) | (SEQ ID NO: 11) |
| (SEQ ID NO: 8) | | | |
| FW3 | CDR3 | FW4 | |
| GVPKRFSGSRSGSDYSLTI | LQYASYPWT | FGGGTKLEIKRADAAP | |
| SSLESEDFVDYYC (SEQ ID | (SEQ ID NO: 13) | TVS | |
| NO: 12) | | (SEQ ID NO: 14) | |

Full-length amino acid sequences of heavy chain and light chain

| | |
|---|---|
| heavy chain | VKLQESGPELKKPGETVKISCKASGYTFTDYSMHWVKQAPGKGLKWMGWINTETGEPTYAD |
| | DFKGRFAFSLETSASTAYLQINNLKNEDTATYFCARTAVYWGQGTTVTVSS (SEQ ID |
| | NO: 15) |
| light chain | DIQMTQSPSSLSASLGERVSLTCRASQEISVSLSWLQQEPDGTIKRLIYATSTLDSGVPKR |
| | FSGSRSGSDYSLTISSLESEDFVDYYCLQYASYPWTFGGGTKLEIKRADAAPTVS (SEQ |
| | ID NO: 16) |

In some embodiments, the anti-EpEX antibody of the present invention is a functional variant of EpAb2-6 which is characterized in comprising (a) a VH comprising HC CDR1 of SEQ ID NO: 2, HC CDR2 of SEQ ID NO: 4, and HC CDR3 of SEQ ID NO: 6; and (b) a V_{L} comprising LC CDR1 of SEQ ID NO: 9, LC CDR2 of SEQ ID NO: 11, and HC CDR3 of SEQ ID NO: 13, or an antigen-binding fragment thereof.

In some embodiments, the anti-EpEX antibody of the present invention, having (a) a VH comprising HC CDR1 of SEQ ID NO: 2, HC CDR2 of SEQ ID NO: 4, and HC CDR3 of SEQ ID NO: 6; and (b) a V_{L} comprising LC CDR1 of SEQ ID NO: 9, LC CDR2 of SEQ ID NO: 11, and HC CDR3 of SEQ ID NO: 13, can comprise a V_{H} comprising SEQ ID NO: 15 or an amino acid sequence substantially identical thereto and a V_{L} comprising SEQ ID NO: 16 or an amino acid sequence substantially identical thereto. Specifically, the anti-EpEX antibody of the present invention includes a V_{H} comprising an amino acid sequence has at least 80% (e.g. 82%, 84%, 85%, 86%, 88%, 90%, 92%, 94%, 95%, 96%, 98%, or 99%) identity to SEQ ID NO:15, and a V_{L} comprising an amino acid sequence has at least 80% (e.g. 82%, 84%, 85%, 86%, 88%, 90%, 92%, 94%, 95%, 96%, 98%, or 99%) identity to SEQ ID NO:16. The anti-EpEX antibody of the present invention also includes any recombinantly (engineered)-derived antibody encoded by the polynucleotide sequence encoding the relevant V_{H} or V_{L} amino acid sequences as described herein.

The term “substantially identical” can mean that the relevant amino acid sequences (e.g., in FRs, CDRs, V_{H}, or V_{L}) of a variant differ insubstantially as compared with a reference antibody such that the variant has substantially similar binding activities (e.g., affinity, specificity, or both) and bioactivities relative to the reference antibody. Such a variant may include minor amino acid changes. It is understandable that a polypeptide may have a limited number of changes or modifications that may be made within a certain portion of the polypeptide irrelevant to its activity or function and still result in a variant with an acceptable level of equivalent or similar biological activity or function. In some examples, the amino acid residue changes are conservative amino acid substitution, which refers to the amino acid residue of a similar chemical structure to another amino acid residue and the polypeptide function, activity or other biological effect on the properties smaller or substantially no effect. Typically, relatively more substitutions can be made in FR regions, in contrast to CDR regions, as long as they do not adversely impact the binding function and bioactivities of the antibody (such as reducing the binding affinity by more than 50% as compared to the original antibody). In some embodiments, the sequence identity can be about 80%, 82%, 84%, 85%, 86%, 88%, 90%, 92%, 94%, 95%, 96%, 98%, or 99%, or higher, between the reference antibody and the variant. Variants can be prepared according to methods for altering polypeptide sequence known to one of ordinary skills in the art such as those found in references which compile such methods, e.g. Molecular Cloning: A Laboratory Manual, J. Sambrook, et al., eds., Second Edition, Cold Spring Harbor Laboratory Press, Cold Spring Harbor, New York, 1989. For example, conservative substitutions of amino acids include substitutions made amongst amino acids within the following groups: (i) A, G; (ii) S, T; (iii) Q, N; (iv) E, D; (v) M, I, L, V; (vi) F, Y, W; and (vii) K, R, H.

The antibodies described herein may be animal antibodies (e.g., mouse-derived antibodies), chimeric antibodies (e.g., mouse-human chimeric antibodies), humanized antibodies, or human antibodies. The antibodies described herein may also include their antigen-binding fragments e.g. a Fab fragment, a F(ab′)₂ fragment, a Fv fragment, a single chain Fv (scFv) and a (scFv)₂. The antibodies or their antigen-binding fragments can be prepared by methods known in the art

More details of an anti-EpEX antibody as used herein are as described in U.S. Pat. No. 9,187,558, the relevant disclosures of each of which are incorporated by reference herein for the purposes or subject matter referenced herein.

Numerous methods conventional in this art are available for obtaining antibodies or antigen-binding fragments thereof.

In some embodiments, the antibodies provided herein may be made by the conventional hybridoma technology. In general, a target antigen e.g. a tumor antigen optionally coupled to a carrier protein, e.g. keyhole limpet hemocyanin (KLH), and/or mixed with an adjuvant, e.g complete Freund's adjuvant, may be used to immunize a host animal for generating antibodies binding to that antigen. Lymphocytes secreting monoclonal antibodies are harvested and fused with myeloma cells to produce hybridoma. Hybridoma clones formed in this manner are then screened to identify and select those that secrete the desired monoclonal antibodies.

In some embodiments, the antibodies provided herein may be prepared via recombinant technology. In related aspects, isolated nucleic acids that encode the disclosed amino acid sequences, together with vectors comprising such nucleic acids and host cells transformed or transfected with the nucleic acids, are also provided.

For examples, nucleic acids comprising nucleotide sequences encoding the heavy and light chain variable regions of such an antibody can be cloned into expression vectors (e.g., a bacterial vector such as an E. coli vector, a yeast vector, a viral vector, or a mammalian vector) via routine technology, and any of the vectors can be introduced into suitable cells (e.g., bacterial cells, yeast cells, plant cells, or mammalian cells) for expression of the antibodies. Examples of nucleotide sequences encoding the heavy and light chain variable regions of the antibodies as described herein are as shown in Table 1. Examples of mammalian host cell lines are human embryonic kidney line (293 cells), baby hamster kidney cells (BHK cells), Chinese hamster ovary cells (CHO cells), African green monkey kidney cells (VERO cells), and human liver cells (Hep G2 cells). The recombinant vectors for expression the antibodies described herein typically contain a nucleic acid encoding the antibody amino acid sequences operably linked to a promoter, either constitutive or inducible. Typical vectors contain transcription and translation terminators, initiation sequences, and promoters useful for regulation of the expression of the nucleic acid encoding the antibody. The vectors optionally contain selection markers for both prokaryotic and eukaryotic systems. In some examples, both the heavy and light chain coding sequences are included in the same expression vector. In other examples, each of the heavy and light chains of the antibody is cloned into an individual vector and produced separately, which can be then incubated under suitable conditions for antibody assembly.

The recombinant vectors for expression the antibodies described herein typically contain a nucleic acid encoding the antibody amino acid sequences operably linked to a promoter, either constitutive or inducible. The recombinant antibodies can be produced in prokaryotic or eukaryotic expression systems, such as bacteria, yeast, insect and mammalian cells. Typical vectors contain transcription and translation terminators, initiation sequences, and promoters useful for regulation of the expression of the nucleic acid encoding the antibody. The vectors optionally contain selection markers for both prokaryotic and eukaryotic systems. The antibody protein as produced can be further isolated or purified to obtain preparations that substantially homogeneous for further assays and applications. Suitable purification procedures, for example, may include fractionation on immunoaffinity or ion-exchange columns, ethanol precipitation, sodium dodecyl sulfate polyacrylamide gel electrophoresis (SDS-PAGE), high-performance liquid chromatography (HPLC), ammonium sulfate precipitation, and gel filtration.

When a full-length antibody is desired, coding sequences of any of the V_{H} and V_{L} chains described herein can be linked to the coding sequences of the Fc region of an immunoglobulin and the resultant gene encoding a full-length antibody heavy and light chains can be expressed and assembled in a suitable host cell, e.g., a plant cell, a mammalian cell, a yeast cell, or an insect cell.

Antigen-binding fragments can be prepared via routine methods. For example, F(ab′)₂ fragments can be generated by pepsin digestion of an full-length antibody molecule, and Fab fragments that can be made by reducing the disulfide bridges of F(ab′)₂ fragments. Alternatively, such fragments can also be prepared via recombinant technology by expressing the heavy and light chain fragments in suitable host cells and have them assembled to form the desired antigen-binding fragments either in vivo or in vitro. A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

One antibody can be further modified to conjugate one or more additional elements at the N- and/or C-terminus of the antibody such as another protein and/or a drug or carrier. Preferably, an antibody conjugated with an additional element retains the desired binding specificity and therapeutic effect while providing additional properties resulted from the additional element that aids, for example, in solubility, storage or other handling properties, cell permeability, half-life, reduction in hypersensitivity, controls delivery and/or distribution. Other embodiments include the conjugation of a label e.g. a dye or fluorophore for assays, detection, tracking and the like. In some embodiments, an antibody can be conjugated to an additional element such as a peptide, dye, fluorophore, carbohydrates, anti-cancer agent, lipid, etc. In addition, an antibody can be attached to the surface of a liposome directly via an Fc region, for example, to form immunoliposomes.

In some embodiments, the second inhibitory agent (an HGFR inhibitor) blocks binding of HGF to HGFR.

In some embodiments, the second inhibitory agent (an HGFR inhibitor) is a small-molecule tyrosine kinase receptor inhibitory compound of HGFR. Table 2 shows some examples of small-molecule HGFR inhibitory compounds.

TABLE 2

| | |
|---|---|
| 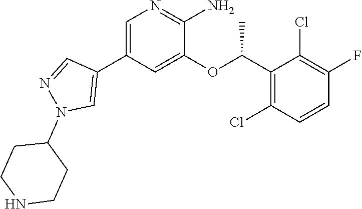 [SMILES: C[C@@H](Oc1cc(-c2cnn(C3CCNCC3)c2)cnc1N)c1c(Cl)ccc(F)c1Cl] | Crizotinib 3-[(1R)-1-(2,6-dichloro-3-fluorophenyl)ethoxy]-5- (1-piperidin-4-ylpyrazol-4-yl)pyridin-2-amine |
| 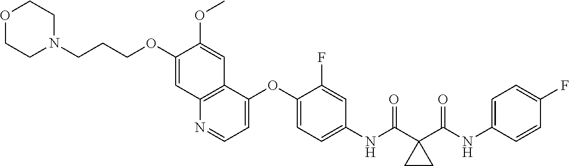 [SMILES: COc1cc2c(Oc3ccc(NC(=O)C4(C(=O)Nc5ccc(F)cc5)CC4)cc3F)ccnc2cc1OCCCN1CCOCC1] | Foretinib N¹-[3-Fluoro-4-({6-methoxy-7-[3-(morpholin-4- yl)propoxy]quinolin-4-yl}oxy)phenyl]-N′¹-(4- fluorophenyl)cyclopropane-1,1-dicarboxamide |
| 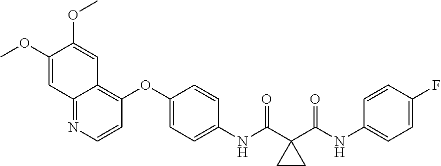 [SMILES: COc1cc2nccc(Oc3ccc(NC(=O)C4(C(=O)Nc5ccc(F)cc5)CC4)cc3)c2cc1OC] | Cabozantinib 1-N-[4-(6,7-dimethoxyquinolin-4-yl)oxyphenyl]- 1-N′-(4-fluorophenyl)cyclopropane-1,1- dicarboxamide |
| 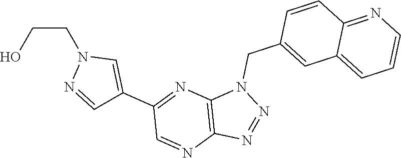 [SMILES: OCCn1cc(-c2cnc3nnn(Cc4ccc5ncccc5c4)c3n2)cn1] | PF-04217903 2-(4-(3-(quinolin-6-ylmethyl)-3H-[1,2,3]triazolo [4,5-b]pyrazin-5-yl)-1 H-pyrazol-1-yl)ethanol |
| 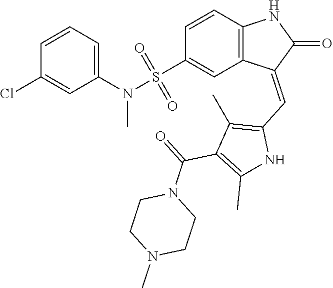 [SMILES: Cc1[nH]c(/C=C2/C(=O)Nc3ccc(S(=O)(=O)N(C)c4cccc(Cl)c4)cc32)c(C)c1C(=O)N1CCN(C)CC1] | SU11274 (3Z)-N-(3-Chlorophenyl)-3-({3,5-dimethyl-4- [(4-methylpiperazin-1-yl)carbonyl]-1H-pyrrol-2- yl}methylene)-N-methyl-2-oxo-2,3- dihydro-1H-indole-5-sulfonamide |

Additional examples of HGFR inhibitory compounds useful in the present invention include but are not limited to AMEP (Bioalliance), EMD-1204831 (Merck KgaA/EMD Serono), INCB-028060 (Incyte/Novartis), ARQ197 (ArQule), AMG102 (Amgen) and RG-3638 (Roche/Genentech). Details are described in WO2012042421A1, for example, which is herein incorporated by reference in their entirety.

As used herein the term “small-molecule HGFR inhibitory compound” or “small-molecule HGFR inhibitor” may include a small-molecule compound that inhibits or binds to HGFR. Unless indicated otherwise, all references herein to small-molecule HGFR inhibitors include references to pharmaceutically acceptable salts, solvates, hydrates and complexes thereof, and to solvates, hydrates and complexes of pharmaceutically acceptable salts thereof, including polymorphs, stereoisomers, and isotopically labeled versions thereof.

As used herein, the term “pharmaceutically acceptable salt” includes acid addition salts. “Pharmaceutically acceptable acid addition salts” refer to those salts which retain the biological effectiveness and properties of the free bases, which are formed with inorganic acids such as hydrochloric acid, hydrobromic acid, sulfuric acid, nitric acid, phosphoric acid and the like, and organic acids such as acetic acid, propionic acid, pyruvic acid, maleic acid, malonic acid, succinic acid, fumaric acid, tartaric acid, citric acid, benzoic acid, mandelic acid, methanesulfonic acid, ethanesulfonic acid, p-toluenesulfonic acid, salicylic acid, trifluoroacetic acid and the like. The term “pharmaceutically acceptable salt” also includes base salts. Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

The term “effective amount” used herein refers to the amount of an active ingredient to confer a desired biological effect in a treated subject or cell. The effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience.

A subject to be treated by the method of treatment as described herein can be a mammal, more preferably a human. Mammals include, but are not limited to, farm animals, sport animals, pets, primates, horses, dogs, cats, mice and rats.

As used herein, “pharmaceutically acceptable carrier” means that the carrier is compatible with an active ingredient in the composition, and preferably can stabilize said active ingredient and is safe to the receiving individual. Said carrier may be a diluent, vehicle, excipient, or matrix to the active ingredient. Typically, a composition comprising an active ingredient e.g. an EpCAM inhibitor, a HGFR inhibitor or a combination thereof can be formulated in a form of a solution such as an aqueous solution e.g. a saline solution or it can be provided in powder form. Appropriate excipients also include lactose, sucrose, dextrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may further contain pharmaceutically acceptable auxiliary substances as required to approximate physiological conditions, for example, pH adjusting and buffering agents, such as sodium acetate, sodium chloride, potassium chloride, calcium chloride, sodium lactate and the like. The form of the composition may be tablets, pills, powder, lozenges, packets, troches, elixers, suspensions, lotions, solutions, syrups, soft and hard gelatin capsules, suppositories, sterilized injection fluid, and packaged powder. The composition of the present invention may be delivered via any physiologically acceptable route, such as oral, parenteral (such as intramuscular, intravenous, subcutaneous, and intraperitoneal), transdermal, suppository, and intranasal methods. In certain embodiments, the composition of the present invention is administered as a liquid injectable formulation which can be provided as a ready-to-use dosage form or as a reconstitutable stable powder.

In some embodiments, the two active components used in the present invention, an EpCAM inhibitor and a HGFR inhibitor, may be formulated as a mixture or independently, in kit form, for simultaneous, separate or sequential administration to a subject. Each component may be formulated together with a suitable pharmaceutically acceptable carrier for proper administration routes. In some embodiments, an EpCAM inhibitor and an HGFR inhibitor may be provided in suitable packaging units where an EpCAM inhibitor or a composition comprising the same and an HGFR inhibitor or a composition comprising the same are present within distinct packaging units.

According to the present invention, combined use of an EpCAM inhibitor and an HGFR inhibitor provides synergistic effects in treating cancer, particularly in inhibiting migration/invasion of cancer cells, reducing tumor size, reducing or suppressing tumor progression, metastasis, and/or prolonging survival of a cancer patient, as compared with the EpCAM inhibitor or the HGFR inhibitor alone. In particular, as shown in the examples (e.g. Example 2.8), in the metastatic and orthotopic animal models, all animals in the control IgG and HGFR inhibitor (crizotinib) groups exhibit significant tumors and poor survival, while the group treated by an EpCAM traizin antibody (EpAb2-6) as an EpCAM inhibitor exhibits slower tumor progression and higher median survival, and surprisingly the combination treatment using an EpCAM neu antibody (EpAb2-6) as an EpCAM inhibitor and an HGFR inhibitor (crizotinib) provides synergistic pronounced effect in reducing tumor progression.

In some embodiments, an EpCAM inhibitor and an HGFR inhibitor are administered simultaneously, separately or sequentially to provide a synergistic anticancer or anti-metastasis effect and in particular the cancer is sensitive to the synergistic combination.

In some embodiments, the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

EpCAM signaling is known to promote colon cancer progression and metastasis. While metastasis is one of the main causes of cancer treatment failure, the involvement of EpCAM signaling in metastatic processes is unclear. Here, we demonstrate that the soluble extracellular domain of EpCAM (EpEX) binds to HGFR and induces downstream signaling in colon cancer cells. We also show that EpEX production is elevated upon HGF treatment and that EpEX and HGF cooperatively regulate HGFR signaling. Moreover, EpEX enhances the metastatic potential of colon cancer cells by activating ERK and FAK-AKT signaling pathways, and it further stabilizes active β-catenin and Snail proteins by decreasing GSK3β activity. Finally, we show that the combined treatment of anti-EpCAM neutralizing antibody (EpAb2-6) and an HGFR inhibitor (crizotinib) significantly inhibits tumor progression and prolongs survival in metastatic and orthotopic animal models of colon cancer. Our findings illuminate the molecular mechanisms underlying EpCAM signaling promotion of colon cancer metastasis, further suggesting the combination of EpAb2-6 and crizotinib may be an effective strategy for colon cancer treatment.

### 1. Material and Methods

### 1.1 Chemicals and Antibodies

Anti-α-tubulin and GAPDH antibodies were from Sigma-Aldrich. Antibodies against human EpCAM, total ERK and Thr202/Tyr204-phosphorylated ERK, total AKT, Ser473-phosphorylated AKT, total HGFR, Tyr1234/1235-phosphorylated HGFR, Non-phospho (Active) β-Catenin (Ser45), β-Catenin, E-cadherin, Vimentin, Snail, Slug, and Twist were from Cell Signaling Technology. LY294002 (AKT inhibitor) was also from Cell Signaling Technology. Foretinib (HGFR inhibitor), SU11274 (HGFR inhibitor), U0126 (MEK inhibitor), PF-562271 (FAK inhibitor), and BIO (GSK3 beta inhibitor) were obtained from Selleck Chemicals. Crizotinib (HGFR inhibitor) was obtained from Med Chem Express. Antibodies against total GSK3 beta, phosphorylated GSK3 beta (phospho S9), phosphorylated ADAM17 (phospho T735), total ADAM17, phosphorylated presenilin 2/AD5 (phospho S327), total presenilin 2/AD5, V5-tag, 6× His-tag, and c-Myc-tag, as well as the Met (pY1234/pY1235)+total Met ELISA Kit (ab126451) were obtained from Abcam. Human HGFR (c-MET) and HGF recombinant proteins were obtained from Sino Biological.

### 1.2 Cell Lines and Culture

The following human cell lines were used: HEK293T, colorectal cancer cell line HCT116 (ATCC: CCL-247), and HT29. The cells were cultivated in Dulbecco modified Eagle's media (DMEM) supplemented with 10% fetal bovine serum (FBS; Gibco) and 100 μg/ml Penicillin/Streptomycin (P/S; Gibco) at 37° C. in a humidified incubator with 5% CO2.

1.3 Mammalian Lentiviral shRNA

For knockdown experiments, human EpCAM shRNAs in the pLKO vector were obtained from the RNAi core facility at Academia Sinica. Lentivirus was produced according to standard protocols with minor modifications. In brief, 293T cells were seeded at a density of 70% in a 100-mm dish and transfected with packaging vectors (i.e., pCMV-ΔR8.91, containing gag, pol and rev genes), envelope vectors (i.e., pMD2.G; VSV-G expressing plasmid), and an individual shRNA vector. The shRNA plasmids were transfected into 293T cells using poly-jet transfection reagent (SignaGen Laboratories). After an overnight incubation, the medium was changed to BSA-containing media. HCT116 cells were infected with viral supernatant containing polybrene (8 μg/ml) for 24 h. The infection procedure was repeated, and then cells were incubated in puromycin (2 μg/ml) for 7 days to select those with stable shRNA expression.

### 1.4 EpCAM Gene Knockout

For the EpCAM knockout, CRISPR/cas9 gRNA constructs were purchased from Genescript. To produce the lentivirus, 293T cells were transiently transfected with CRISPR/cas9 gRNA plasmids, the EpCAM gRNA (target sequence: GTGCACCAACTGAAGTACAC, SEQ ID NO: 21), packaging plasmid (pCMV-ΔR8.91) and an envelope expression plasmid (pMD.G). HCT116 or HT29 cells were cultured with lentivirus-containing medium and selected with 2 μg/ml puromycin. Single cell clones were isolated from the selected pool, and the expression of EpCAM was examined with Western blotting.

### 1.5 Production and Purification of EpEX-his Recombinant Protein

Recombinant protein was expressed and purified using the Expi293 expression system. Cells were grown in Expi293 expression medium, and protein expression was induced by addition of enhancer reagent. Supernatant was harvested by centrifugation After centrifugation at 8000 g for 20 min at 4° C., the supernatant was incubated with nickel-chelated affinity resin (Ni-NTA, Qiagen) for 2 h at 4° C. The resin was washed with wash buffer, containing 50 mM Tris-HCl (pH 8.0), 500 mM NaCl, and 20 mM imidazole, and the proteins were eluted with elution buffer, containing 50 mM Tris-HCl (pH 8.0), 500 mM NaCl, and 250 mM imidazole.

### 1.6 Construction of the EpCAM EGF-Like Domain Deletion Mutant

In its extracellular domain, EpCAM contains two EGF-like domains at amino acids 27-59 (1st EGF-like domain) and 66-135 (2nd EGF-like domain), and a cysteine-free motif (Schnell et al., 2013). The EpCAM EGF-like domain deletion mutant was generated using a standard QuikChange™ deletion mutation system with 1st forward mutagenic deletion primer (5′-GCAGCTCAGGAAGAATCAAAGCTGGCTGCC-3′, SEQ ID NO: 22), 1st reverse mutagenic deletion primer (5′-GGCAGCCAGCTTTGATTCTTCCTGAGCTGC-3′, SEQ ID NO: 23), 2nd forward primer (5′-AAGCTGGCTGCCAAATCTGAGCGAGTGAGA-3′, SEQ ID NO: 24) and 2nd reverse primer (5′-TCTCACTCGCTCAGATTTGGCAGCCAGCTT-3′, SEQ ID NO: 25). The PCR amplifications were performed using KAPA HiFi Hot Start DNA polymerase (Kapa Biosystems), and products were treated with restriction enzyme, DpnI (Thermo Scientific), to digest methylated parental DNAs.

### 1.7 Immunoprecipitation Assay

Cells were lysed in lysis buffer (50 mM Tris-HCl, pH 7.4, 150 mM NaCl, and 1% NP-40) with Protease Inhibitors (Roche). For immunoprecipitation, cell lysates were incubated with antibodies for 6 h at 4° C. Then, 20 pL Dynabeads Protein G was added and the mixture was incubated for 2 h at 4° C. to pull-down the antibody-bound protein. The immunoprecipitation samples were washed with PBS three times, denatured in sample buffer, and analyzed by Western blotting.

### 1.8 Generation of Monoclonal Antibodies and Purification of IgG

Generation of EpAb2-6 and control IgG were performed as described previously (Liao et al., 2015). The experimental protocol was approved by the Committee on the Ethics of Animal Experiments of Academia Sinica (AS IACUC: 11-04-166).

### 1.9 Protein Extraction and Immunoblotting

Whole cell extracts were prepared with RIPA buffer (50 mM Tris-HCl pH7.4, 1% NP-40, 0.5% Sodium deoxycholate, 0.1% SDS, 150 mM NaCl, 2 mM EDTA, 50 mM NaF). Protein concentrations of the cell lysates were determined by the Bradford assay. The lysates were separated on a 10% polyacrylamide gel and then transferred to PVDF membrane. The membrane was blocked for 1 h with 3% BSA in PBST. The membrane was then incubated overnight with primary antibodies.

Appropriate horseradish peroxidase-associated secondary antibodies (Millipore) were applied and the membranes were incubated at room temperature (RT) for 1 h. The protein bands were subsequently visualized with chemiluminescence reagents (Millipore) and detected on a BioSpectrum 600 Imaging system (UVP). The protein level was quantified from band intensity using Gel-Pro analyzer 3.1 (Media Cybernetics).

### 1.10 Cell Viability Assay

Cell viability was assayed by measuring mitochondrial dehydrogenase activity with the WST-1 (4-[3-(4-lodophenyl)-2-(4-nitrophenyl)-2H-5-tetrazolio]-1, 3-benzene disulfonate) assay. Cells were seeded in 96-well plates at a density of 10⁴ cells/well and cultured for 24 h. New culture media with EGF, EpEX, or deglycan-EpEX at the indicated concentrations were added to the cells. At the end of the treatment period, 10 μl of WST-1 proliferation reagent (5 μg/ml) was added to each well and incubated for 1 h at 37° C. Following the incubation, absorbance of each well was detected at 450 nm using a spectrophotometric microplate reader.

### 1.11 Colony Formation Assay

Cells were seeded in 24-well plates (1×10⁴ cells/well) and cultured for 7 days. The cells were then fixed with 4% of formaldehyde and stained with crystal violet solution. After capturing images of the plates, a solution with 0.5% SDS was added to each well, and the plates were incubated for 2 h at room temperature. The relative densities of cells were then determined by measuring the absorbance of the solution at 570 nm using a microplate reader. The experiments were performed in triplicate.

### 1.12 Transwell Migration and Invasion Assay

Cell migration and invasion were assayed with 8-μm pore size Transwell migration chambers (Millicell) without or with 10% matrigel. Cells (1×10⁵) were added to the upper chamber in 500 μl serum-free DMEM. Then, 700 μl DMEM containing 10% FBS was added to the lower chamber as a chemoattractant. Migration and invasion were allowed to proceed for 16 h at 37° C. in a standard cell culture incubator. Then, cells were removed from the upper surface of the membranes with cotton swabs, and the cells that had migrated to the lower surface were stained with 0.05% (w/v) crystal violet in 4% paraformaldehyde (in 1×PBS) for 15 min and washed with water. Membranes were dried for 15-20 min before at least four random fields on the membrane examined at high power were counted for each experimental condition.

### 1.13 Apoptosis Assays

Cells were seeded and treated with 10 μg/ml mAb or inhibitor for 6 h; an unrelated mouse myeloma immunoglobulin used at appropriate dilution served as the IgG2a (Invitrogen #02-6200) isotype control. Apoptotic cells were detected using Annexin V-FITC and PI, and were analyzed using a flow cytometer (BD Immmunocytometry Systems). Early apoptosis was measured with the Annexin V-FITC Apoptosis Detection kit II (BD Pharmingen). Late apoptotic nuclei were detected with propidium iodide (PI) staining.

1.14 RNA Extraction, cDNA Synthesis, Quantitative Reverse Transcription Polymerase Chain Reaction (qRT-PCR)

Total RNA extraction, first strand cDNA synthesis, and SYBR-green based real-time PCR were performed as described in the manufacturer's instructions. To extract total RNA, cells were lysed using TRIzol reagent (Invitrogen), and proteins and phenol were removed from TRIzol using chloroform. After centrifugation, the top colorless layer was collected and mixed with isopropanol to precipitate RNA pellet. The RNA pellet then was washed with 70% ethanol, air-dried at room temperature, and dissolved in RNase free water. For first strand cDNA synthesis, 5 μg of total RNA was used for reverse transcription with oligo(dT) primer and SuperScriptIII reverse transcriptase (Invitrogen) at 50° C. for 60 min. Target gene levels were evaluated by quantitative PCR (qPCR), using LightCycler 480 SYBR Green I Master Mix (Roche) and a LightCycler480 System (Roche). GAPDH mRNA expression was measured as endogenous housekeeping control to normalize all q-PCR reactions. The qPCR reaction was 95° C. for 5 min, followed by 40 cycles of denaturation at 95° C. for 10 s, annealing at 60° C. for 10 s and extension at 72° C. for 30 s. Final results were calculated from three independent experiments. Primer sequences used to detect the mRNA expression of genes of interest are listed in supplementary material Table 3.

TABLE 3

| Gene | Forward, Reverse |
|---|---|
| EPCAM | Forward: 5′- CTCCACGTGCTGGTGTGT R -3′ |
| | (SEQ ID NO: 26) |
| | Reverse: 5′- TGTTTTAGTTCAATGATGATCCAGT |
| | A -3′ (SEQ ID NO: 27) |
| E- | Forward: 5′- GGAACTATGAAAAGTGGGCTTG-3′ |
| cadherin | (SEQ ID NO: 28) |
| | Reverse: 5′- AAATTGCCAGGCTCAATGAC-3′ |
| | (SEQ ID NO: 29) |
| VIM | Forward: 5′-GTTTCCCCTAAACCGCTAGG-3′ |
| | (SEQ ID NO: 30) |
| | Reverse: 5′-AGCGAGAGTGGCAGAGGA-3′ |
| | (SEQ ID NO: 31) |
| SNAIL | Forward: 5′-CTTCGGCTCCAGGAGAGTC-3′ |
| | (SEQ ID NO: 32) |
| | Reverse: 5′-TTCCCACTGTCCTCATCTGAC-3′ |
| | (SEQ ID NO: 33) |
| GAPDH | Forward: 5′-CTTCACCACCATGGAGGAGGC-3′ |
| | (SEQ ID NO: 34) |
| | Reverse: 5′-GGCATGGACTGTGGTCATGAG-3′ |
| | (SEQ ID NO: 35) |

### 1.15 Colon Cancer Metastatic Animal Models

Colon cancer HCT116 or HT29 (5×10⁶ cells/mouse) in PBS were injected into 4-6-week-old female NOD/SCID mice through the tail vein. Mice were then randomly assigned to different treatment groups by body weight. After 3 days, antibodies were administered through tail vein injection twice a week for four consecutive weeks. Crizotinib was administered daily by oral gavage for 5 days per week (treatment for four weeks). For the therapeutic study, tumor-bearing mice were treated with isotype control IgG1 (15 mg/kg), crizotinib (20 mg/kg), EpAb2-6 (15 mg/kg), or crizotinib (20 mg/kg) combined with EpAb2-6 (15 mg/kg). Mouse survival rate were measured. Animal care was carried out in accordance with the guidelines of Academia Sinica, Taiwan. The protocol was approved by the Committee on the Ethics of Animal Experiments of Academia Sinica (AS IACUC: 20-05-1468).

### 1.16 Orthotopic Implantation and Therapeutic Studies

Orthotopic tumor models were created as previously reported (Chen et al., 2020). Briefly, NSCID mice were used for orthotopic implantation of HCT116 cells previously infected with Lenti-luc virus (lentivirus containing luciferase gene). The mice were anesthetized by i.p. injection ofAvertin, 2,2,2-Tribromo-ethanol (Sigma-Aldrich) at a dose of 250 mg/kg. Tumor development was monitored by bioluminescence imaging. For the therapeutic study, tumor-bearing mice were treated with isotype control IgG1 (15 mg/kg), crizotinib (20 mg/kg), EpAb2-6 (15 mg/kg), or crizotinib (20 mg/kg) combined with EpAb2-6 (15 mg/kg). Tumor progression was monitored by quantification of bioluminescence. Mouse survival was also monitored. Animal care was carried out in accordance with the guidelines of Academia Sinica. The protocol was approved by the Committee on the Ethics of Animal Experiments of Academia Sinica (AS IACUC: 20-05-1468).

### 1.17 Statistical Analysis

All data are presented as mean±SEM for the indicated number of experiments. Unpaired Student's t-test was used to analyze the expression percentages in experimental versus control cultures. Ap-value of less than 0.05 was considered statistically significant.

### 2. Results

2.1 EpEX Induces HGFR Phosphorylation Through Interaction with HGFR

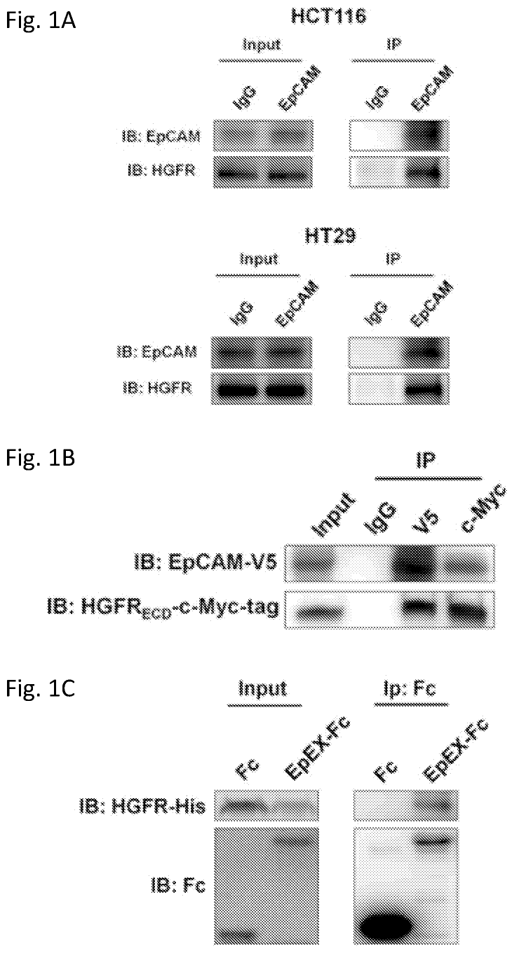

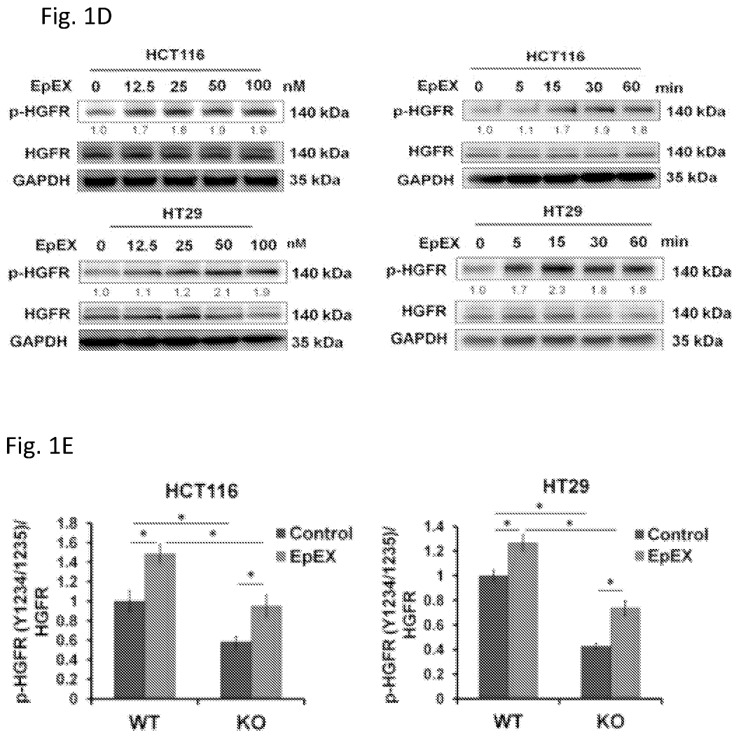

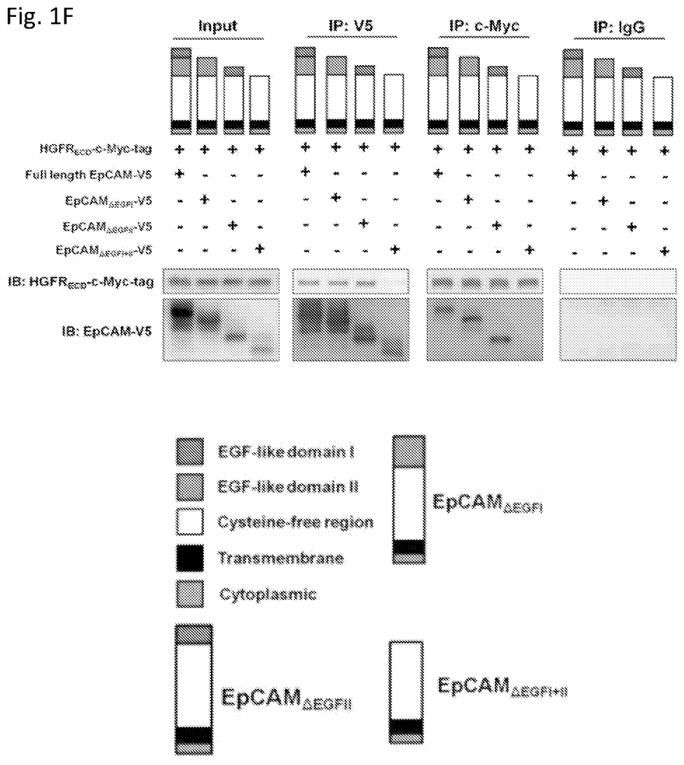

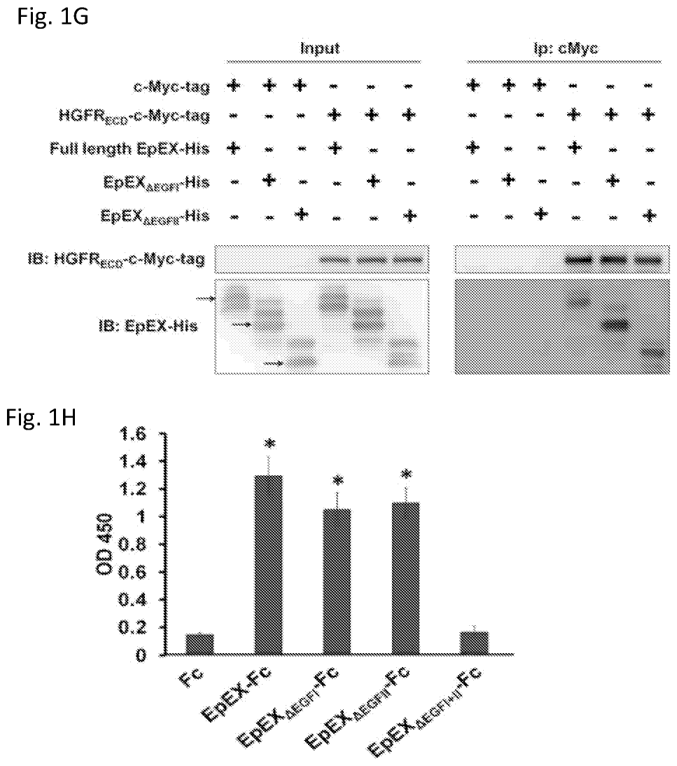

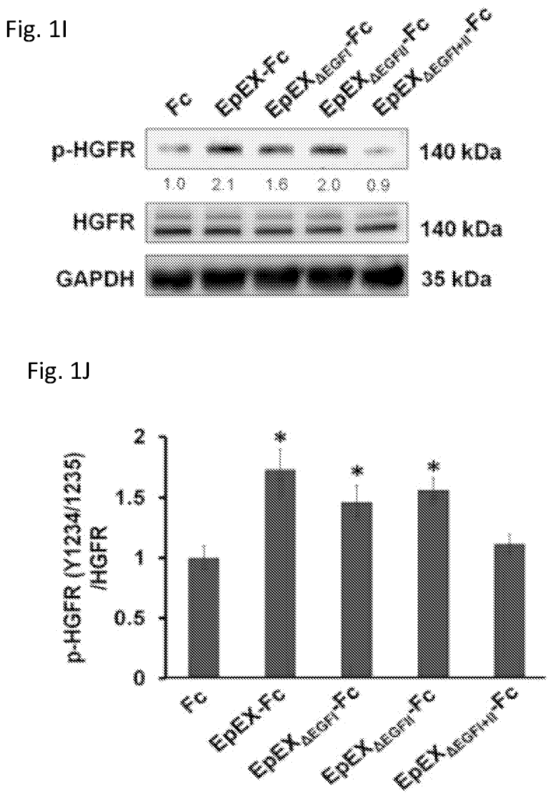

In our previous study, we conducted a Human Phospho-RTK Array Kit (R&D Systems) assay and found that EpEX induces both EGFR and HGFR phosphorylation in HCT116 cells (Liang et al., 2018). To test whether endogenous EpCAM directly interacts with HGFR in HCT116 and HT29 colon cancer cell lines, we used DTSSP, a cross-linker, to stabilize the putative EpCAM-HGFR complex. As we predicted, the interaction of EpCAM and HGFR was confirmed by immunoprecipitation (IP) and Western blotting (FIG. 1A). To further study whether membrane-bound EpCAM could bind to extracellular domain of HGFR (HGFR_{ECD}), we performed co-IP experiments using HEK293T cells that overexpress both EpCAM-V5 and HGFR_{ECD}-c-Myc-tag. The results confirmed interactions between exogenous EpCAM and HGFR (FIG. 1B). Next, we performed IP to probe the direct interaction between recombinant EpEX-Fc and HGFR_{ECD}-His recombinant protein. (FIG. 1C). To investigate the effect of EpEX on the phosphorylation of HGFR in colon cancer cells, we analyzed the levels of phosphorylated HGFR in HCT116 and HT29 cells. The Western blotting and ELISA results showed that both EpCAM and EpEX induced phosphorylation of HGFR in both cell types. In fact, only EpEX could induce HGFR phosphorylation in the absence of EpCAM (FIG. 1D and FIG. 1E).

EpEX is comprised of two EGF-like domains (Schnell et al, 2013), hence we sought to determine which domain interacts with HGFR. To do so, we constructed various EGF-like domain deletion mutants (EpCAM_{ΔEGFI+II}, EpCAM_{ΔEGFI} and EpCAM_{ΔEGFII}) plasmids. Surprisingly, the mutants harboring only one EGF-like domain deletion (EpCAM_{ΔEGFI} and EpCAM_{ΔEGFII}) could interact with HGFR while both domain-deleted mutants (EpCAM_{ΔEGFI+II}) did not show results of such kind (FIG. 1F). A similar result was observed when assessing HGFR_{ECD} binding with soluble EpEX wild-type or mutant proteins (FIG. 1G). Overall, these findings indicate that membrane-bound EpCAM and secreted EpEX can both bind HGFR through either EGF domain I or II of EpCAM/EpEX.

Next, we performed ELISA to probe the potential interactions between several variants of EGF-like-domain-deleted mutants of EpEX-Fc and HGFR-His proteins. The results confirmed EpEX binding to HGFR via its both domain when HGFR binding to EpEX_{ΔEGFI+II} mutant protein was completely abolished (FIG. 1H). Similar to previous phosphorylation results exhibited by wild-type EpEX (FIGS. 1D and E), we observed both EpEX_{ΔEGFI} and EpEX_{ΔEGFII} could induce HGFR phosphorylation which however EpEX_{ΔEGFI+II} protein could not (FIG. 1I and FIG. 1J). Based on these results, we conclude that EpEX can bind to HGFR and induce consequent phosphorylation.

### 2.2 EpEX Promotes Tumor Progression Through HGFR Signaling

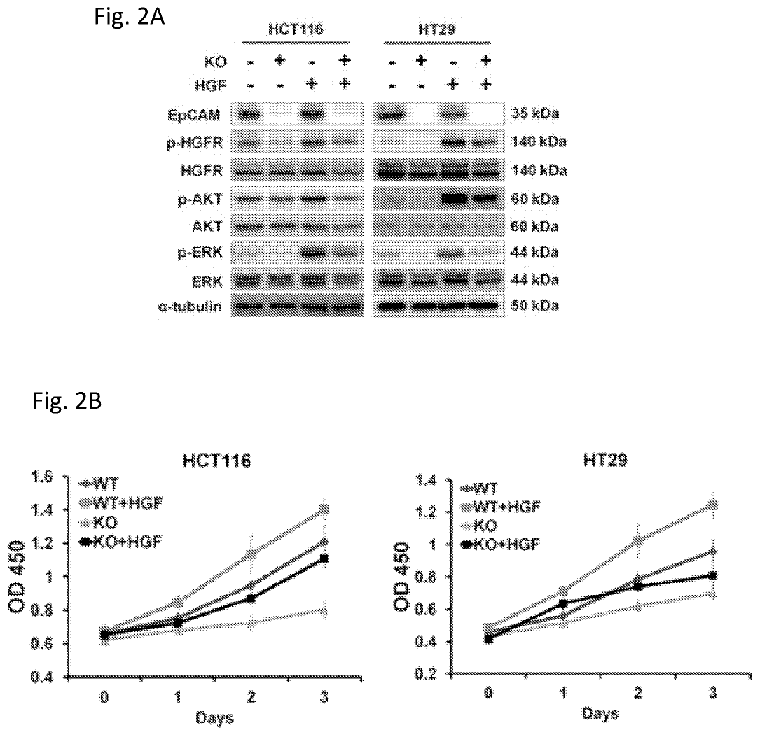

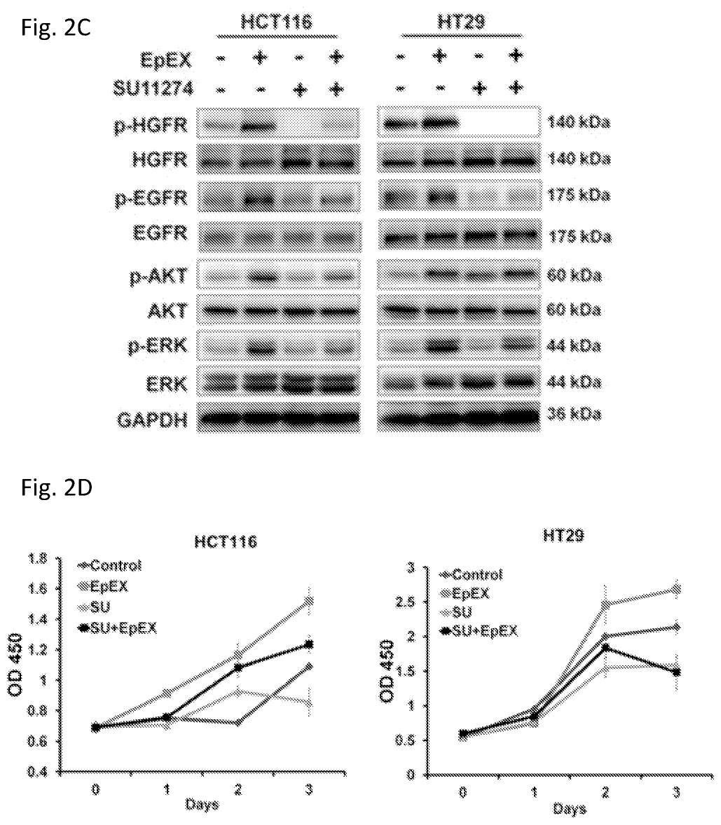

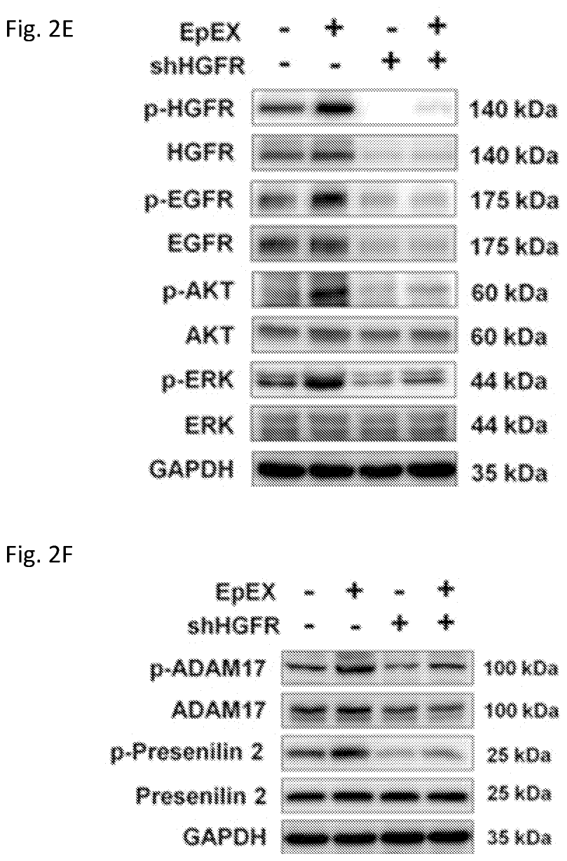

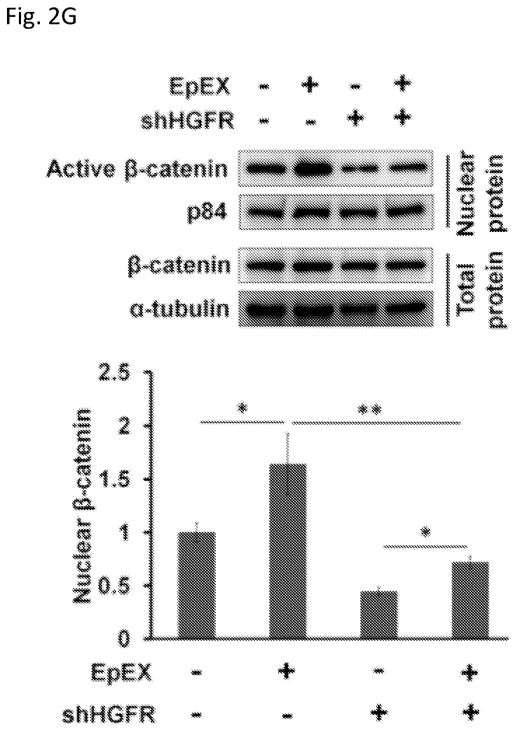

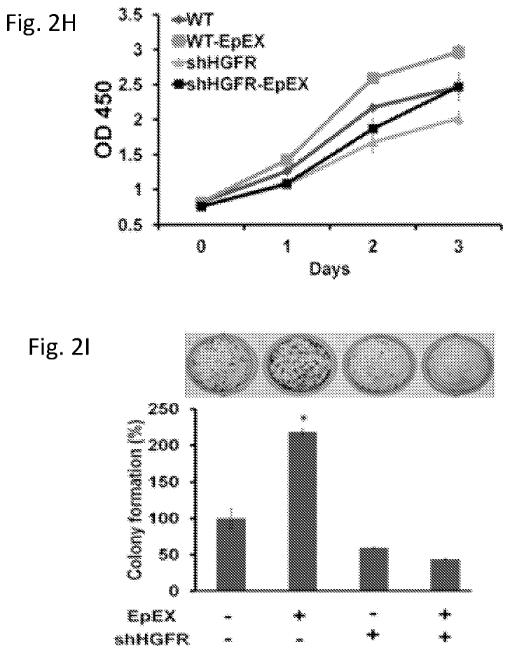

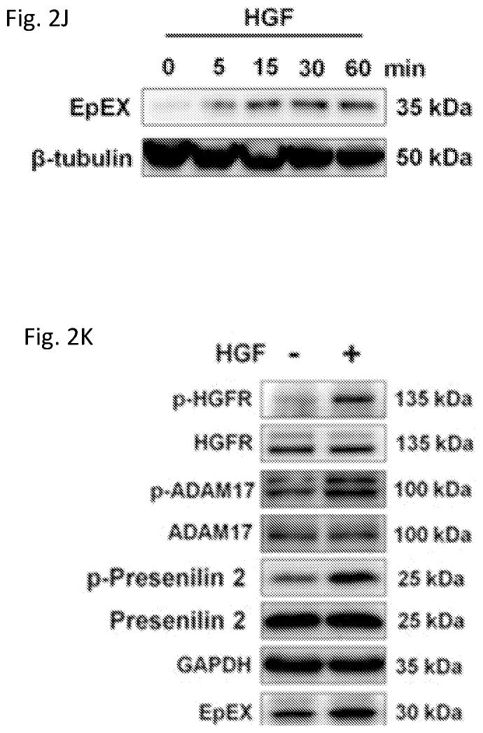

The ability of EpCAM to induce HGFR phosphorylation suggested certain possibilities that this pathway might be partially responsible for tumorigenicity in colon cancer cells. To investigate whether EpCAM and HGFR activation cooperatively regulate cancer progression and metastasis, we examined the levels of phosphorylated ERK and AKT in EpCAM knockout cells with or without HGF treatment. Western blotting showed that phosphorylation of HGFR, AKT and ERK are not affected by treatment with HGF in EpCAM knockout HCT116 and HT29 cells (FIG. 2A). Further, results of the cell growth assay showed that EpCAM knockout cells grew slower than wild-type HCT116 and HT29 cells, but the trajectory of cell growth could be restored by treating EpCAM knockout HCT116 and HT29 cells with HGF (FIG. 2B).

To determine whether EpEX could induce cancer progression and invasion via HGFR signaling, we analyzed phosphorylation of EGFR, ERK and AKT in colon cancer cells after treatment with combinations of EpEX-His and SU11274, a tyrosine kinase inhibitor of HGFR. We found that SU11274 could attenuate EpEX-mediated ERK and AKT phosphorylation in HCT116 and HT29 cells (FIG. 2C). Further understanding the effects of such inhibition on cell growth, we found that EpEX treatment alone stimulated growth of HCT116 and HT29 cells, while SU11274 abolished the EpEX-induced increases in cell viability and proliferation in colon cancer cells (FIG. 2D).

Furthermore, HGFR knockdown reduced phosphorylation levels in HGFR, EGFR, AKT, and ERK. Interestingly, HGFR knockdown also reduced the level of EGFR phosphorylation (FIG. 2E). Moreover, HGFR knockdown diminished EpEX-induced RIP (phosphorylated ADAM17 and presenilin 2) (FIG. 2F) and nuclear translation of active β-catenin (FIG. 2G). In addition, both EpEX-induced cell growth and colony formation were significantly reduced by HGFR knockdown in HCT116 cells (FIG. 2H and FIG. 2I). In fact, we found that EpEX production was elevated after HGF treatment of HCT116 cells using an IP assay (FIG. 2J). We also noticed HGF treatment increased the phosphorylation of ADAM17, presenilin 2 and EpEX production in HCT116 cells (FIG. 2K).

### 2.3 EpEX Activates ERK and FAK-AKT Signaling

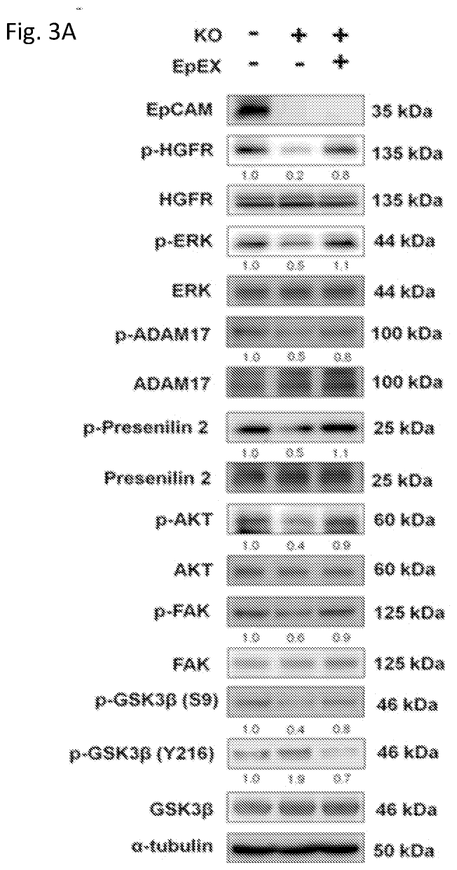

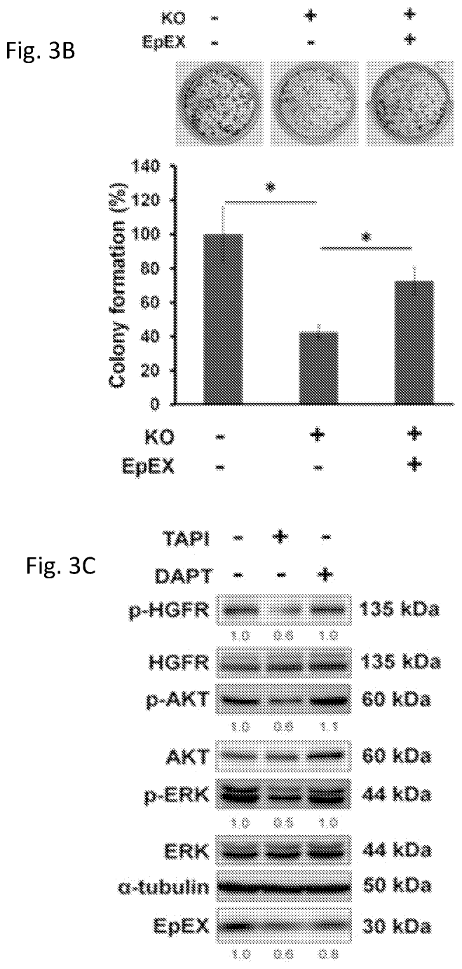

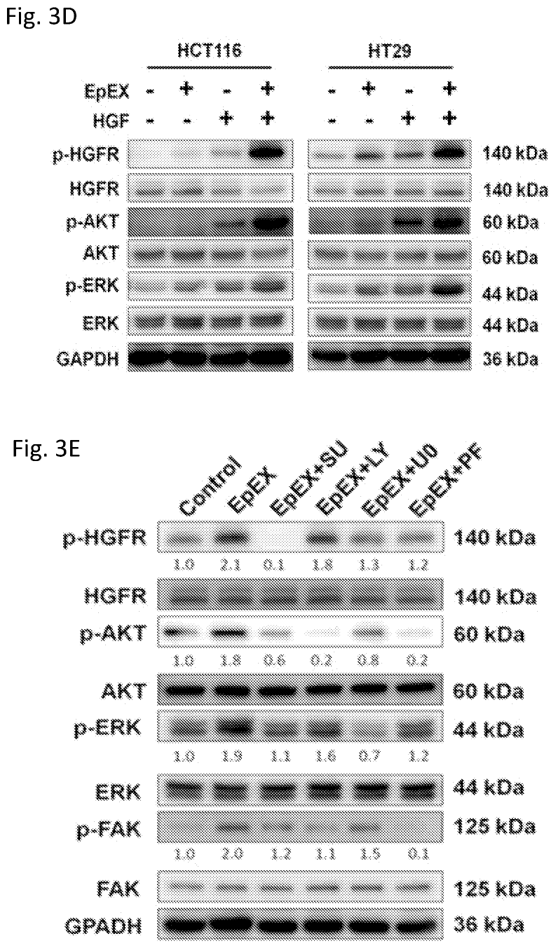

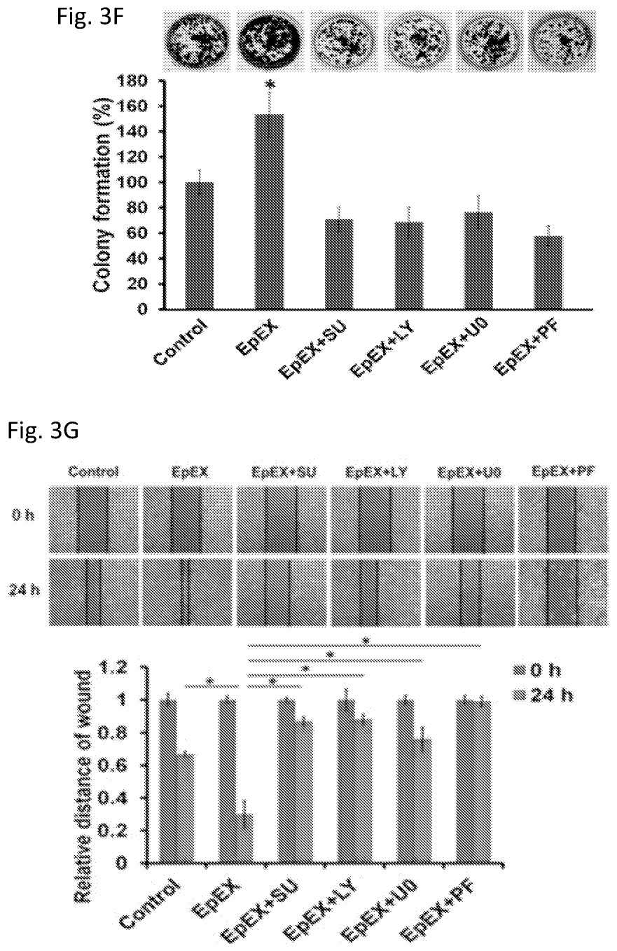

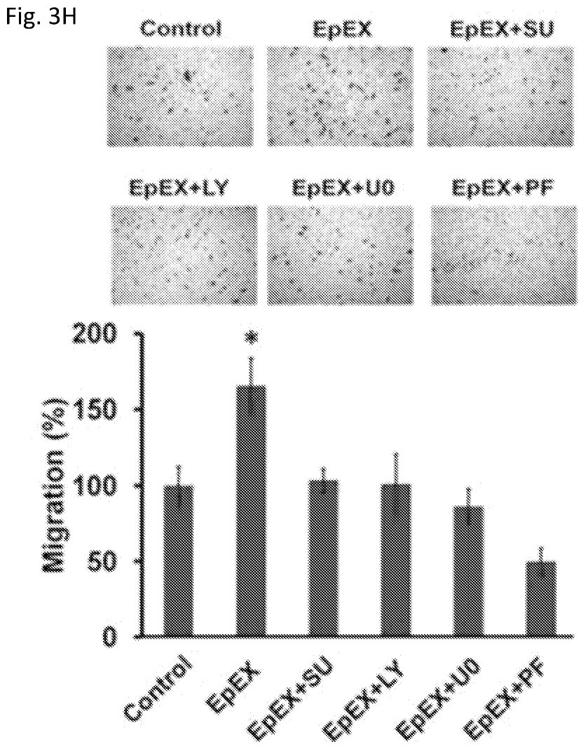

EpCAM is known to influence the growth, survival and metastasis of cancer cells via its downstream effectors. In its process of signaling, the proteolysis of EpCAM produces EpEX, which may further stimulate RIP and release of EpICD that subsequently transduces EpCAM signaling (Lin et al, 2012). Moreover, it was previously shown that EpEX treatment to HCT116 cells can increase RIP via phosphorylation of TACE and presenilin 2, the catalytic subunit of γ-secretase (Liang et al., 2018). We therefore treated EpCAM knockout HCT116 cells with EpEX that resulted partial restoration of HGFR downstream signaling such as phosphorylation of AKT, FAK, and ERK as well as phosphorylation of RIP proteins (ADAM17 and presenilin 2) (FIG. 3A). Previous work showed that GSK3β antagonists stimulate EMT via AKT (An et al, 2020). In the line of this mechanism, we found that EpEX could rescue suppressive phosphorylation of GSK3β (S9, inactive GSK3β), while it simultaneously decreased activating phosphorylation of GSK3β (Y216, active GSK3β) in EpCAM knockout cells (FIG. 3A). We also found that EpEX increased colony formation in EpCAM knockout HCT116 cells (FIG. 3B), and blocking the shedding of endogenous EpEX but not EpICD, caused decreased HGFR, AKT and ERK phosphorylation suggesting the endogenous EpEX was crucial for HGFR signaling activation (FIG. 3C).

We next tested whether EpEX could enhance HGF induced HGFR signaling. Incubation of HCT116 and HT29 cells with soluble EpEX in combination with HGF upregulated phosphorylation of HGFR and subsequent downstreams including AKT and ERK compared to EpEX or HGF treatments alone (FIG. 3D). Since EpEX can trigger HGFR activation, we further investigated the effects of EpEX on the mediators of HGFR signaling. In this context, AKT and ERK signaling are two of the most important cancer-associated signaling pathways (Chang et al, 2013; Sun et al, 2015), as they play a variety of physiological roles in the regulation of EMT, cell cycle, survival, and cancer progression. Therefore, we tested whether an HGFR inhibitor (SU11274), AKT inhibitor (LY294002), ERK inhibitor (U0126) and FAK inhibitor (PF-562271) could affect EpEX-induced signaling in HCT116 cells. We noticed that EpEX increased the AKT, ERK and FAK phosphorylation levels (FIG. 3E), colony formation potentials (FIG. 3F), wound healing (FIG. 3G) and migration (FIG. 3H) abilities. Together, these results indicate that EpEX increases ERK and FAK-AKT signaling pathway by inducing HGFR activation in colon cancer cells.

### 2.4 EpEX Promotes EMT and Invasion by Inducing Active β-Catenin and Snail Expression Via Down-Regulating GSK3β Activity

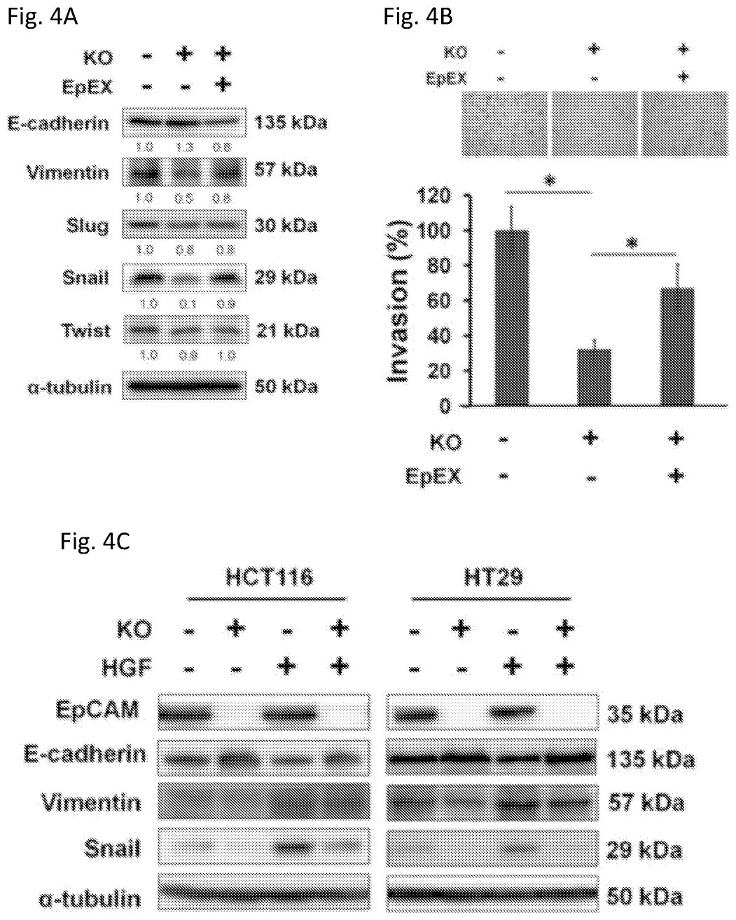

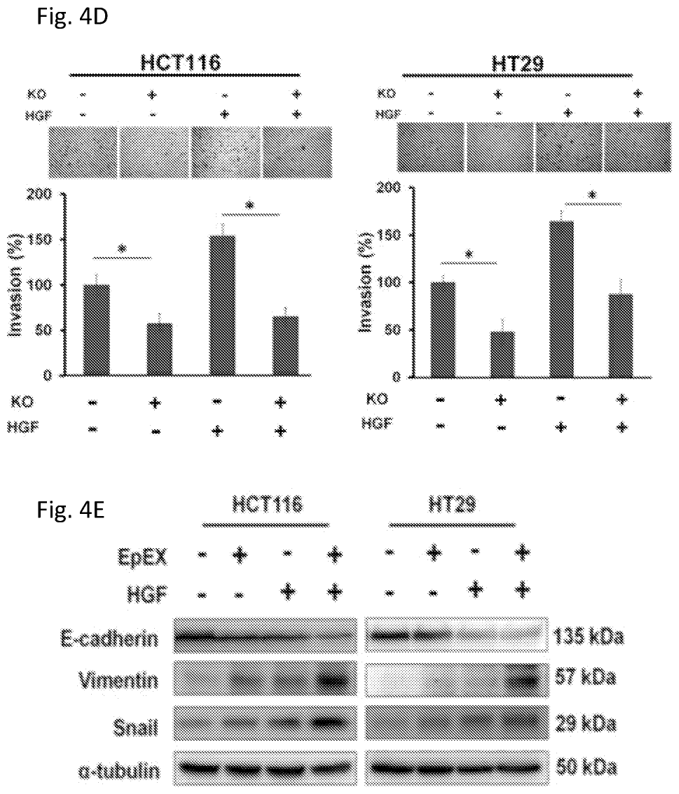

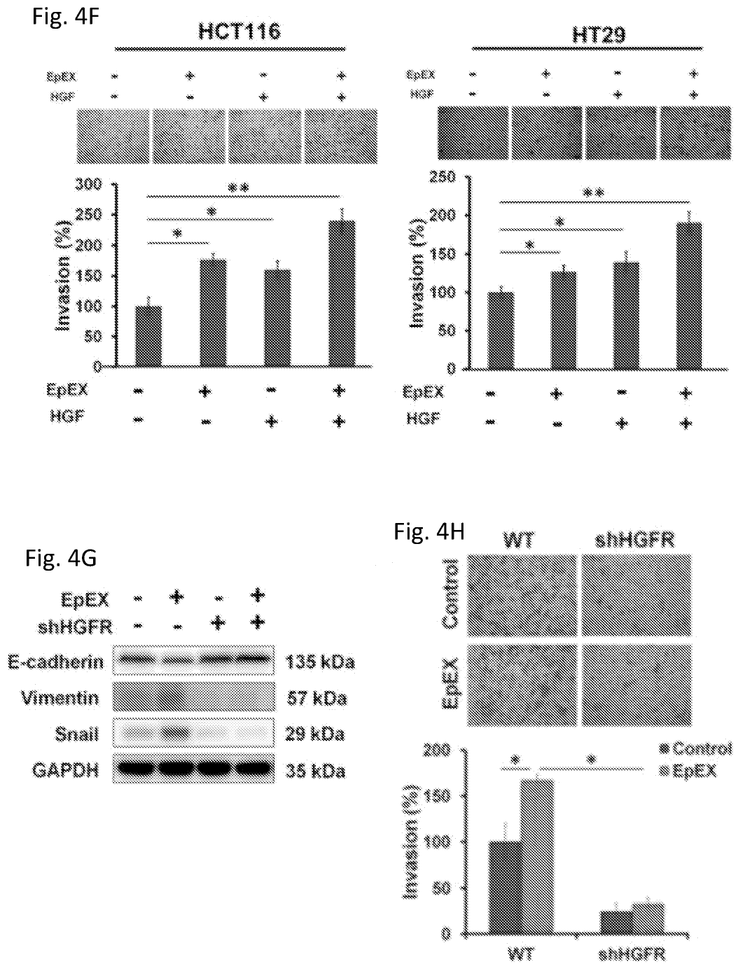

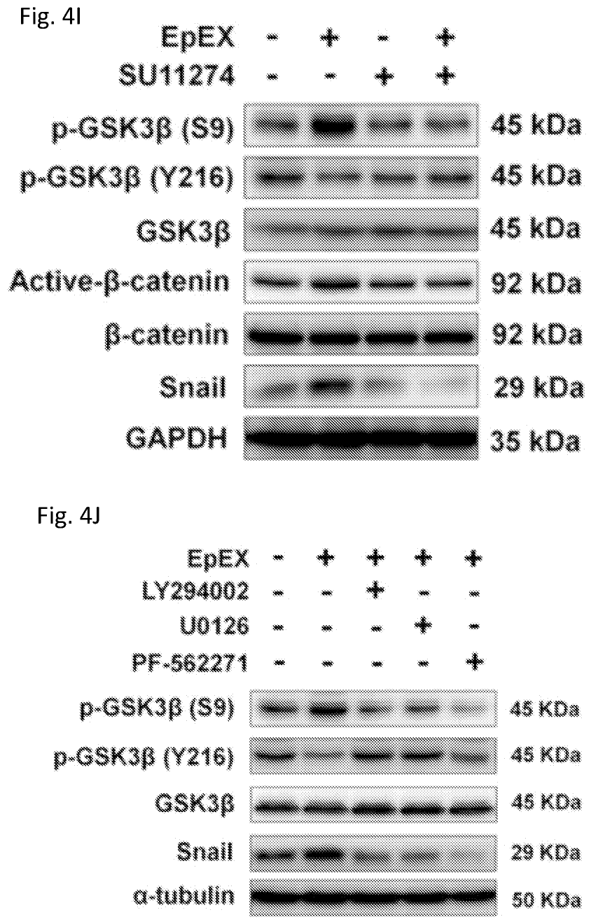

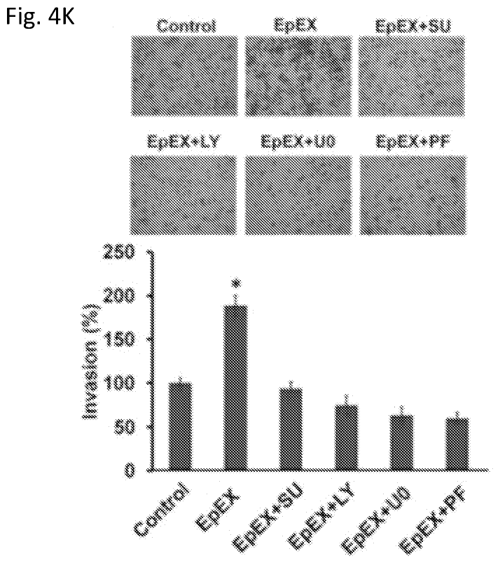

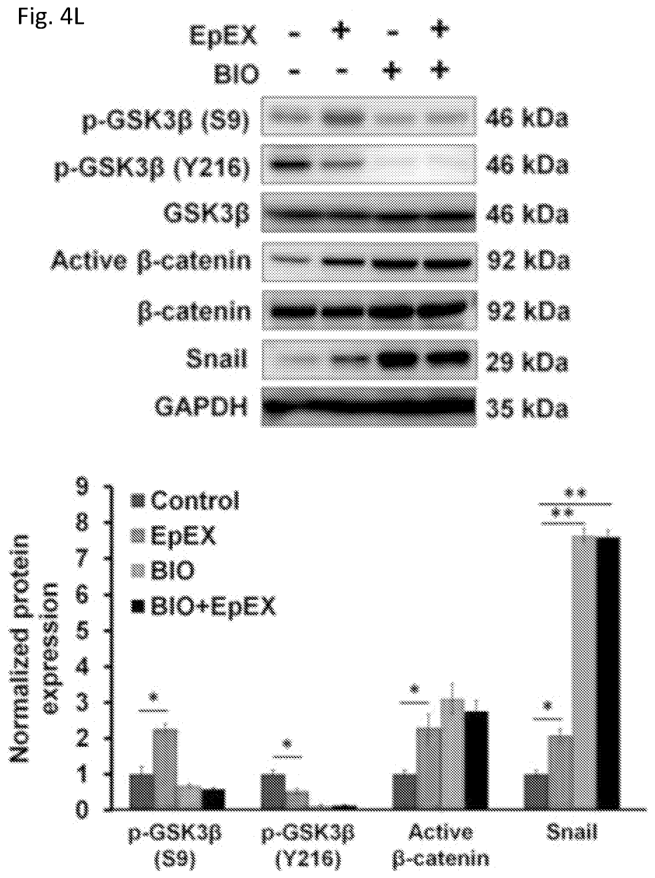

We found that EpCAM knockout inhibited the expressions of the mesenchymal marker vimentin, as well as the protein level of the EMT regulator Snail, while enhancing E-cadherin expression in HCT116 cells; moreover, such EMT indictors were restored after EpEX treatment (FIG. 4A). EpEX also induced cell invasion in EpCAM knockout HCT116 cells (FIG. 4B).

To investigate whether EpCAM and HGFR activation cooperatively regulate cancer cell invasion, we examined EpCAM knockout cells with or without HGF treatment. We found that expression of EMT-related proteins as well as cell invasion properties in EpCAM knockout HCT116 and HT29 cells were significantly reduced in compared to wild-type cells. HGF-induced EMT and invasion activity were also reduced in EpCAM knockout cells (FIG. 4C and FIG. 4D). Incubation of HCT116 and HT29 cells with EpEX in combination with HGF upregulated the levels of EMT and cell invasion compared to EpEX or HGF treatments alone (FIG. 4E and FIG. 4F). Furthermore, EpEX-induced EMT-related protein expressions and cell invasion were prevented by HGFR knockdown (FIG. 4G and FIG. 4H). These results suggest that EpEX can enhance HGFR activation and induces EMT and metastasis in colon cancer cells.

Previous reports indicated that GSK3β antagonists stimulate EMT by AKT signaling thus affect Snail protein turnover via its phosphorylation and ubiquitin-mediated proteolysis (An et al., 2020). In the line of this mechanism, we found that EpEX could induce suppressive phosphorylation of GSK3β (S9, inactive GSK3β), while it simultaneously decreased activating phosphorylation of the protein (Y216, active GSK3β); these changes were coincident with increased Snail protein expression in HCT116 cells. However, SU11274 could attenuate EpEX-mediated GSK3β activity and abolish EpEX-induced active β-catenin and Snail protein expression in HCT116 cells (FIG. 4I). We also found that inhibitors of HGFR downstream mediators (i.e., LY294002, U0126 and PF-562271) could attenuate EpEX-mediated GSK3β activity and Snail protein expression (FIG. 4J). EpEX-induced invasion was also suppressed by LY294002, U0126 and PF-562271 (FIG. 4K). Of note, active β-catenin and Snail protein expressions were upregulated both in control and EpEX-treated cells after treatment with the GSK3β inhibitor, BIO (FIG. 4L). These results suggest that EpEX promotes EMT and invasion by inducing active β-catenin and Snail protein expression via down-regulation of GSK3β activity.

### 2.5 EpEX Promotes Snail Protein Stability Through Inhibition of Ubiquitination-Mediated Proteasomal Degradation

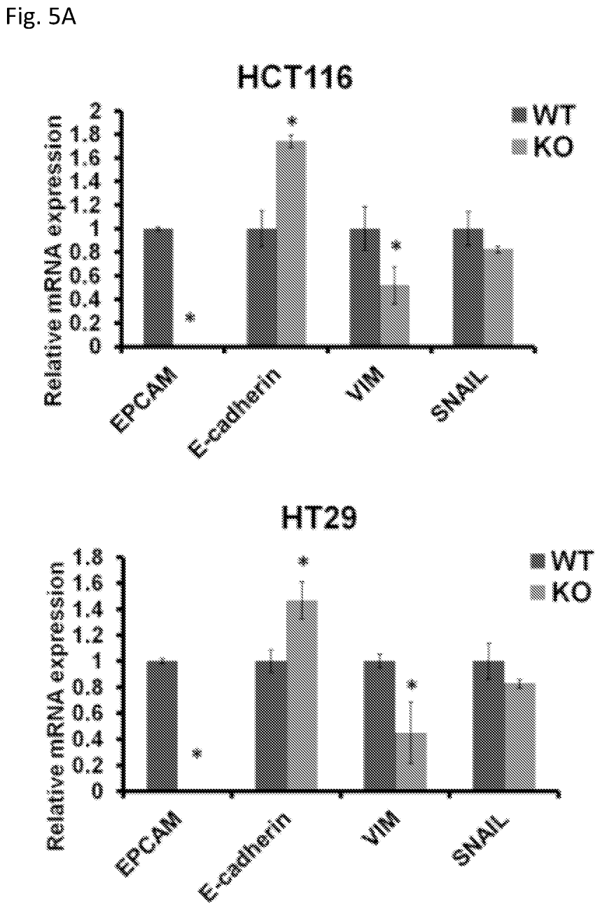

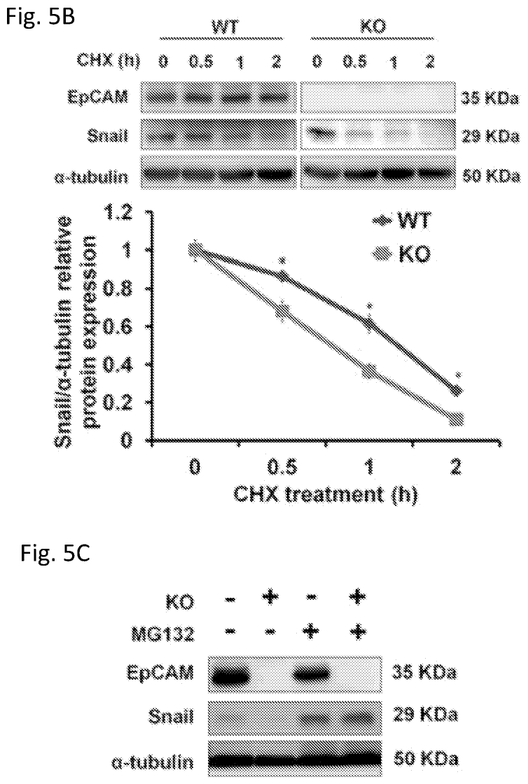

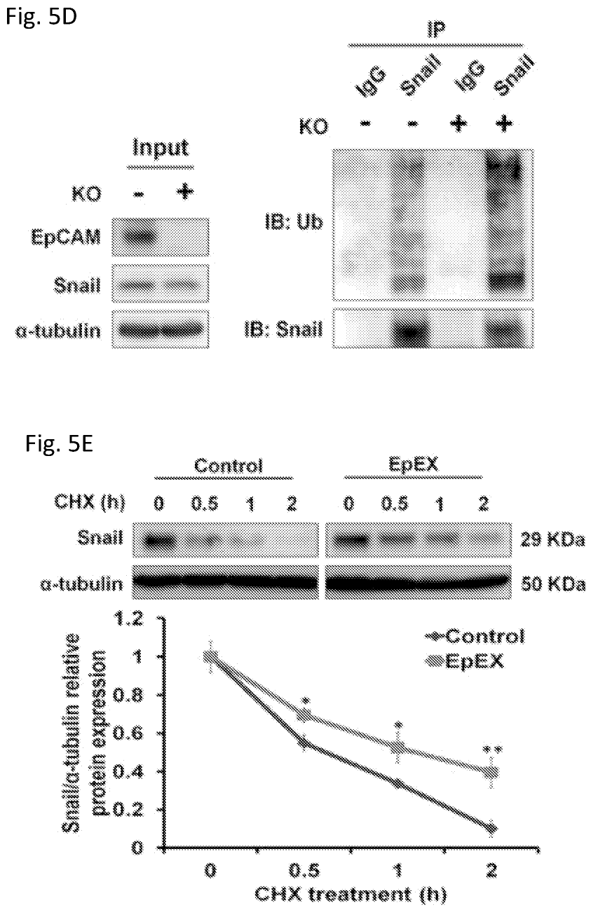

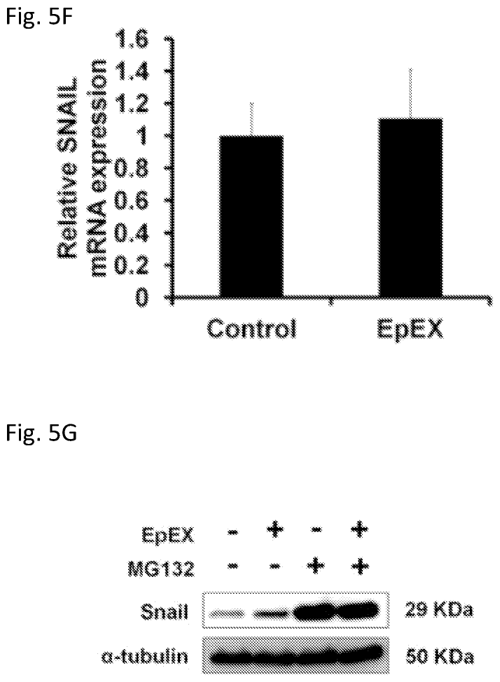

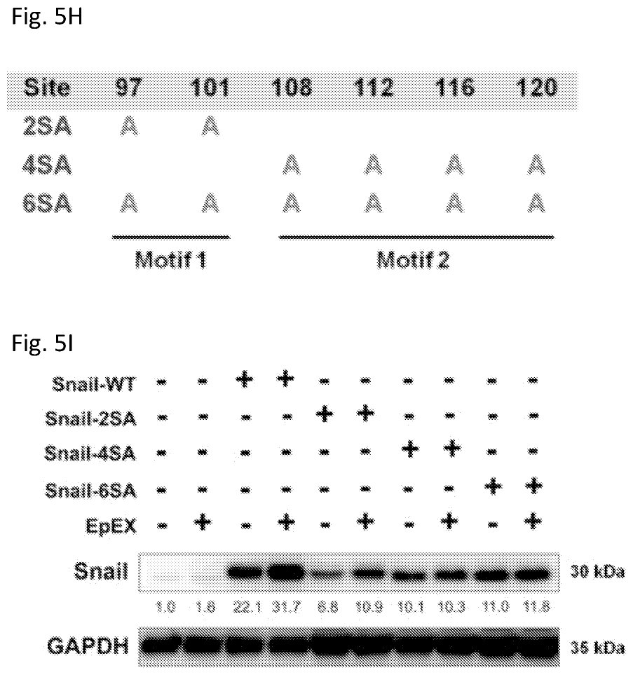

We analyzed the effects of EpCAM on EMT-related gene expression by transfecting colon cancer cells with lentivirus expressing Cas9 and a sgRNA targeting EpCAM. The results showed EpCAM knockout increased the gene expression of E-cadherin and reduced the gene expression of VIM. However, EpCAM knockout did not affect SNAIL gene expression levels (FIG. 5A). In fact, we noticed that cyclohexamide treatment shortened the half-life of Snail protein with knockout of EpCAM (FIG. 5B). On the other hand, treatment with MG132 (an inhibitor of the 26S proteasome) increased Snail steady-state protein levels, indicating the protein level is largely controlled by proteasomal degradation (FIG. 5C). EpCAM knockout also led to an increased level of ubiquitylated Snail compared to that of the control cells (FIG. 5D). Moreover, cyclohexamide treatment further confirmed that EpEX extended Snail protein half-life (FIG. 5E). EpEX did not affect the level of Snail gene expression (FIG. 5F), and MG132 increased Snail steady-state protein levels in HCT116 cells with or without EpEX treatment (FIG. 5G).

In this regard, two consensus motifs in serine-rich regions of Snail (motif 1: S97, S101; motif 2: S108, S112, S116, S120) are crucial to its post-transcriptional regulation and ubiquitination-mediated proteasomal degradation (Zhou et al, 2004). HCT116 cells were transfected with wild-type (Snail-W T) and three mutant (Snail-2SA, -4SA, and -6SA) Snail constructs (FIG. 5H). While treatment of the transfected cells with EpEX significantly increased expression of Snail-WT and -2SA, no such effects were seen on expression of Snail-4SA and -6SA mutants (FIG. 5I). These data suggest that EpEX regulates protein stability of Snail via the serine-rich consensus motif 2 of Snail in cancer cells. Taken together, the results of these experiments revealed that EpEX plays an important role in the regulation of EMT by promoting Snail protein stability in colon cancer cells.

### 2.6 EpAb2-6 Inhibits EpCAM and HGFR Signaling and Promotes Active β-Catenin and Snail Protein Degradation Via Activation of GSK3β

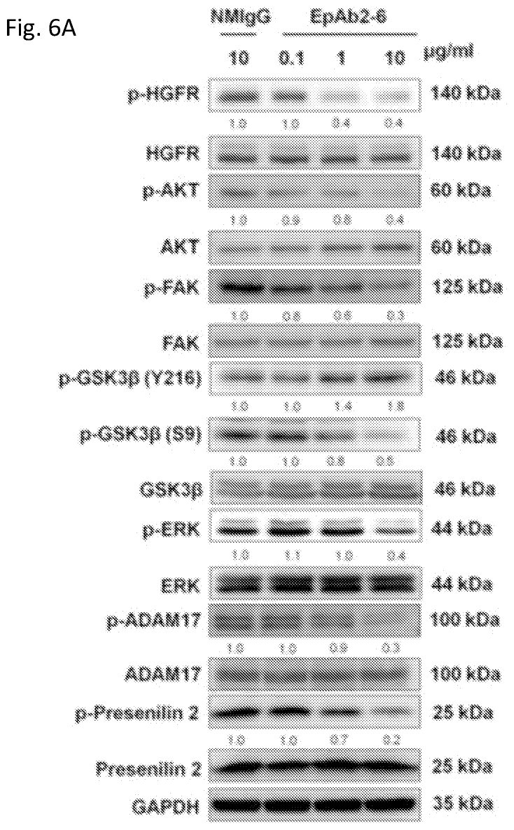

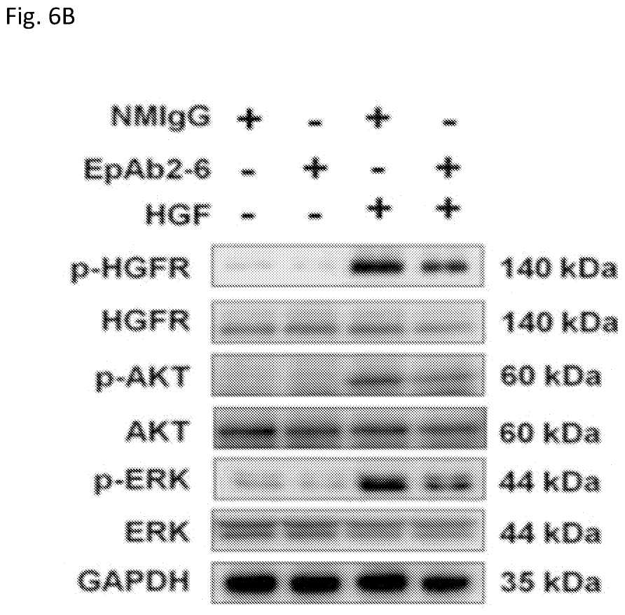

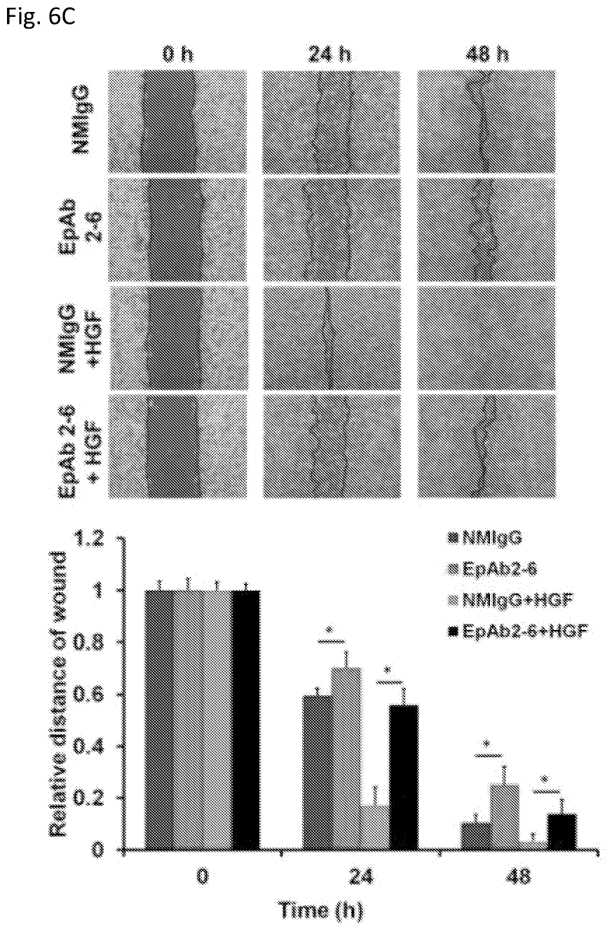

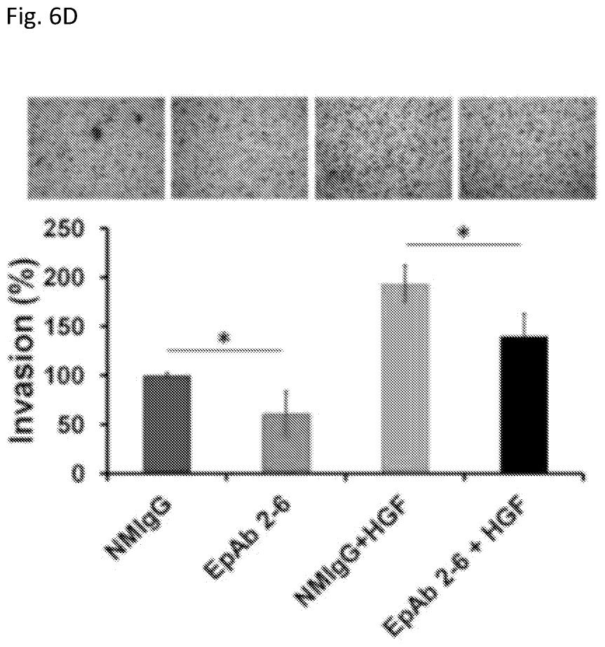

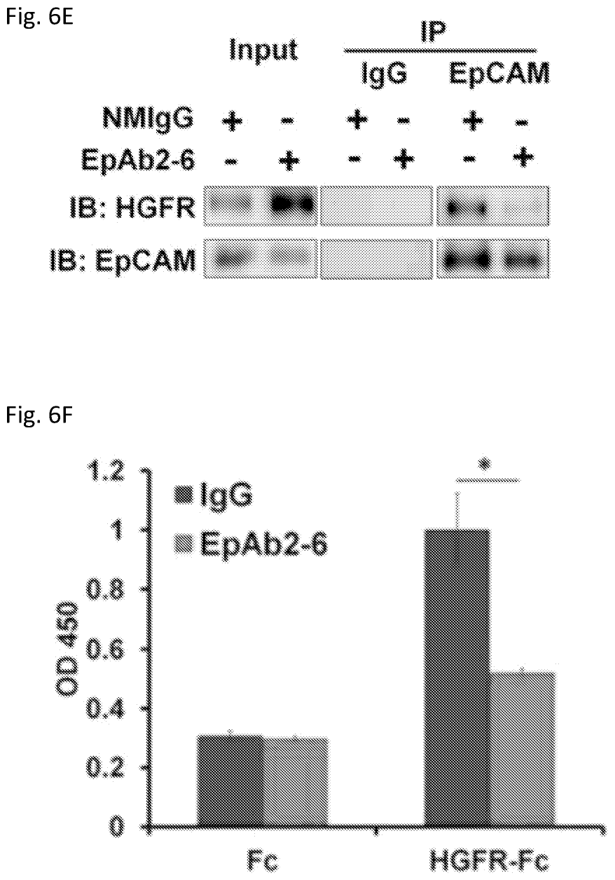

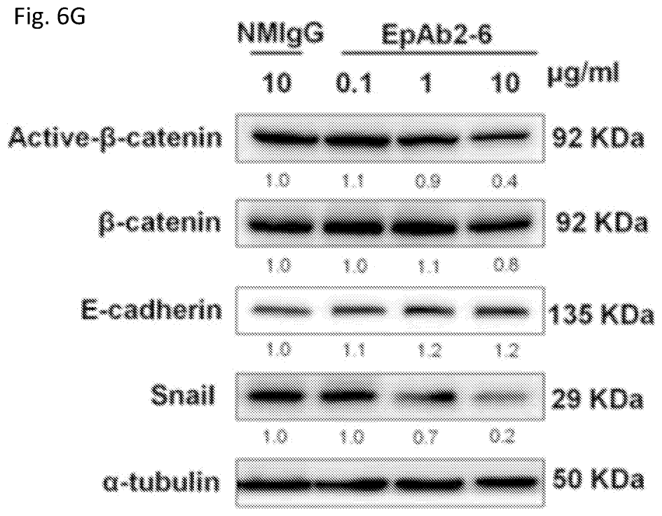

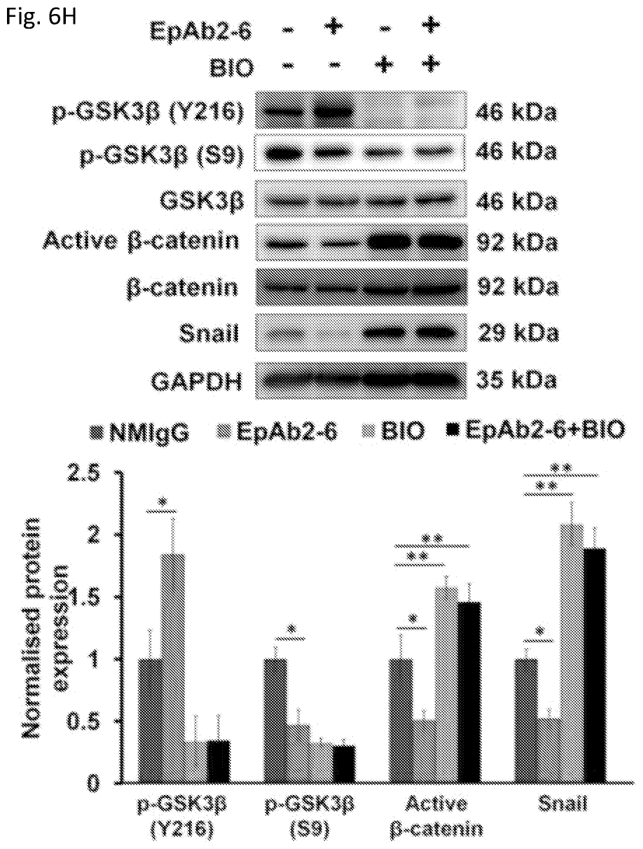

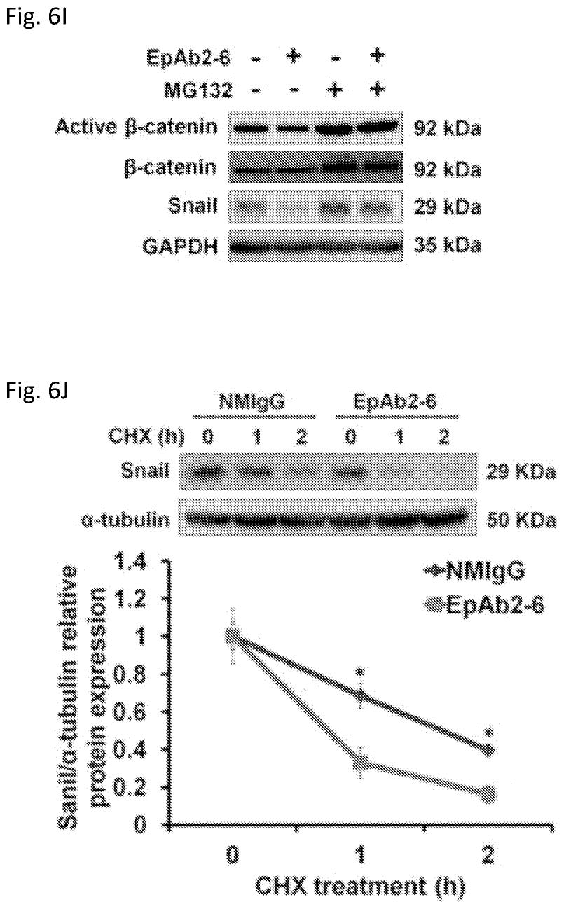

Previously, we developed a neutralizing antibody, EpAb2-6, which targets EpEX and induces cancer cell apoptosis (Liang et al., 2018; Liao et al., 2015). Therefore, we used EpAb2-6 to block the function of EpEX in colon cancer cells and analyzed the phosphorylation levels of HGFR, AKT, FAK, GSK3β, ERK, ADAM17, and presenilin 2 in HCT116 cells. EpAb2-6 treatment resulted decreased phosphorylation of HGFR, AKT, ERK, and FAK in comparison to control IgG treatment (FIG. 6A). HGF treatment increased phosphorylation levels of HGFR, AKT, and ERK in HCT116 cells. Meanwhile, the levels of these phosphorylated proteins were significantly decreased in cells treated with EpAb2-6. Moreover, the invasion and migration activities of HCT116 cells were also significantly reduced with EpAb2-6 treatment (FIG. 6B). When HCT116 cells were treated with HGF after EpAb2-6, the effects of EpAb2-6 on invasion and migration were partially blunted (FIGS. 6C and 6D).

Interestingly, the EpAb2-6 decreased the association between EpCAM and HGFR, as detected by IP of endogenous proteins in HCT116 cells (FIG. 6E). To evaluate whether recombinant EpEX directly binds to HGFR, we performed ELISA to probe the interaction between purified EpEX-His and HGFR-Fc protein. Binding activity of EpEX to HGFR was further confirmed by ELISA, and we also showed that anti-EpCAM monoclonal antibody EpAb2-6 could inhibit EpEX binding to HGFR (FIG. 6F).

Following these experiments, we next analyzed the levels of EMT protein expression in HCT116 cells treated with of control IgG or EpAb2-6. The results showed that EpAb2-6 increased the levels of E-cadherin, while decreasing Snail and the active β-catenin (FIG. 6G). Furthermore, EpAb2-6 decreased suppressive phosphorylation of GSK3β at S9 (inactive GSK3β), and it simultaneously increased activating phosphorylation of the protein at Y216 (active GSK3β). These changes are expected to increase GSK3β activity and were coincident with the observed decreases in active β-catenin and Snail proteins. Correspondingly, active β-catenin and Snail proteins were increased after treatment with the GSK3β inhibitor, BIO (FIG. 6H). Active β-catenin and Snail steady-state protein levels were reduced by treatment with EpAb2-6, while treatment with proteasome inhibitor (MG132) increased active β-catenin and Snail steady-state protein levels (FIG. 6I). In addition, EpAb2-6 shortened the Snail protein half-life, as shown in a cyclohexamide treatment assay (FIG. 6J). These results suggest that EpAb2-6 inhibits metastatic processes by downregulating HGFR signaling and allows active β-catenin and Snail protein degradation via increased GSK3β activity.

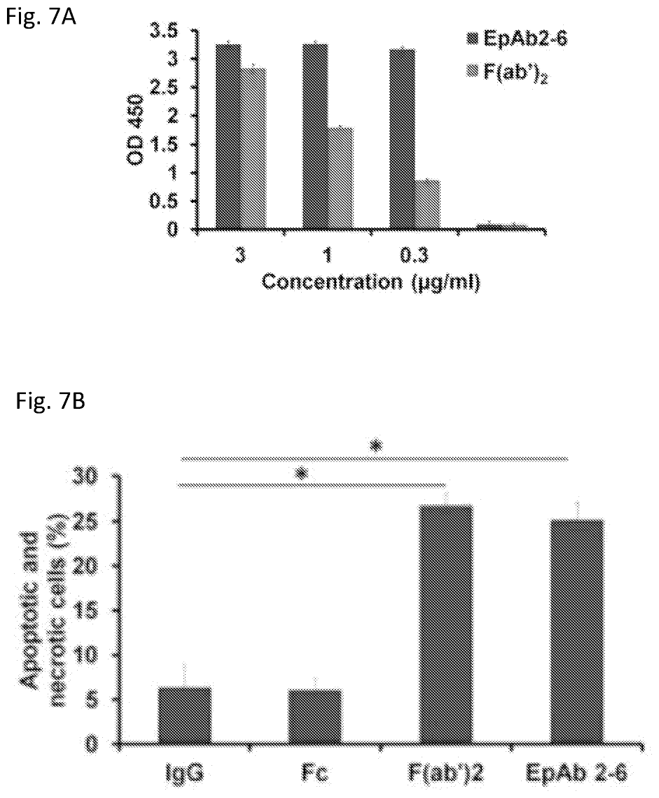

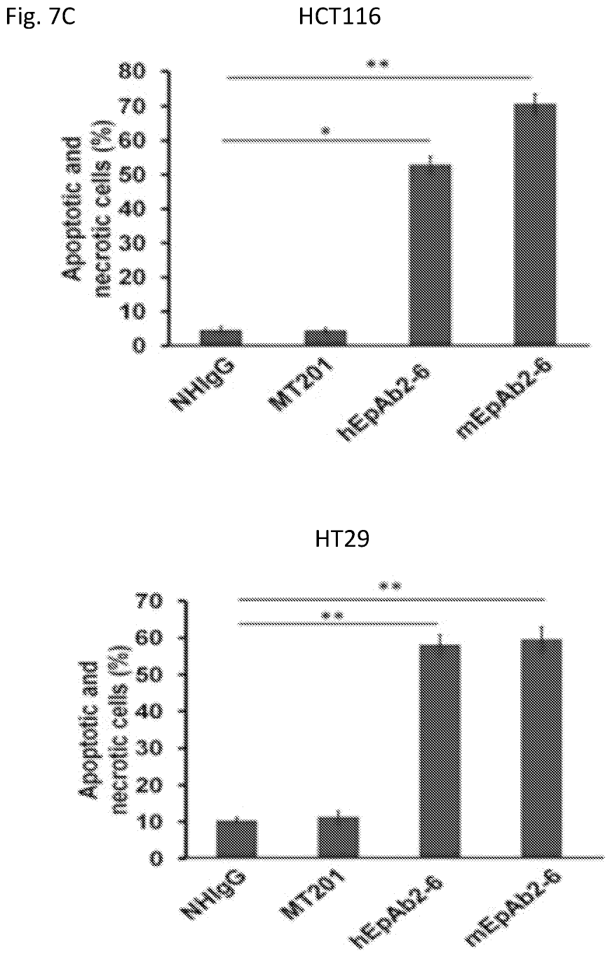

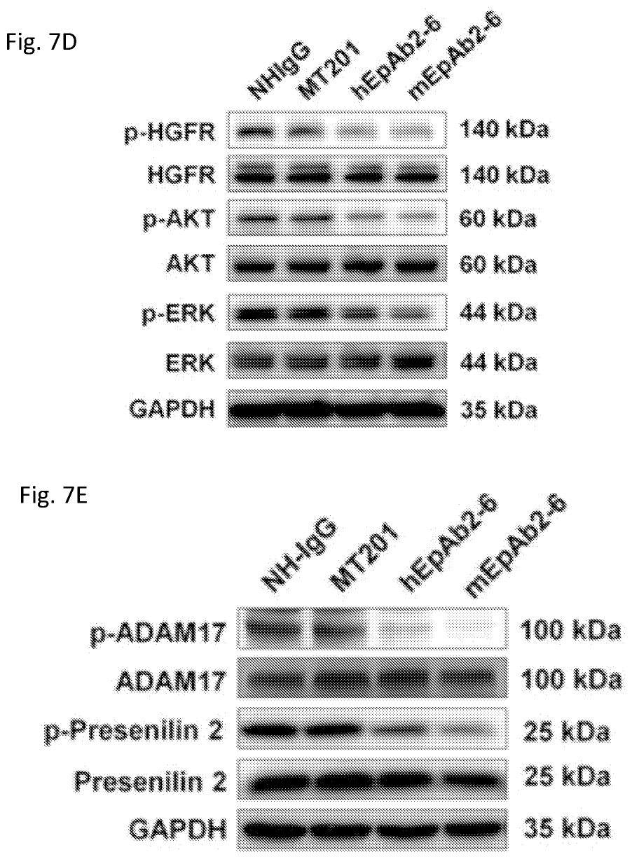

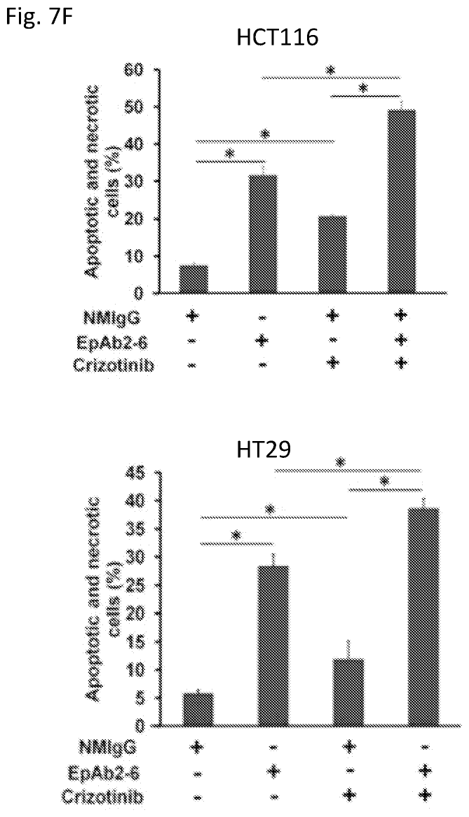

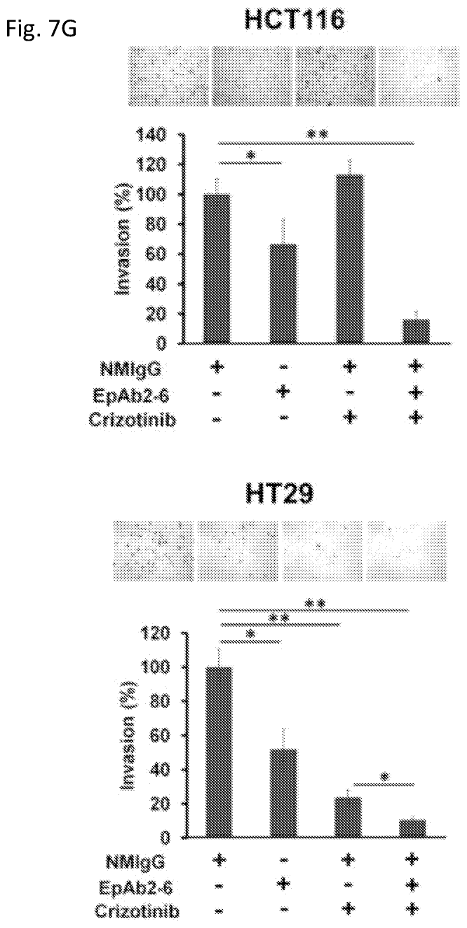

We further tested whether divalent antibody fragments F(ab′)₂ of EpAb2-6 bind EpEX could induce apoptosis. The results showed that F(ab′)₂ of EpAb2-6 could indeed bind to EpEX (FIG. 7A) and induce apoptosis in colon cancer cells (FIG. 7B). We also used an apoptosis assay to evaluate whether humanized EpAb2-6 (hEpAb2-6) and human anti-EpCAM antibody, adecatumumab (MT201), share similar activities. EpAb2-6 and hEpAb2-6 exhibited similar functional attributes in terms of inducing apoptosis, while MT201 did not show such effects in HCT116 or HT29 cancer cells (FIG. 7C). We also found that EpAb2-6 and hEpAb2-6 both inhibited phosphorylation of HGFR, AKT, and ERK, but MT201 did not (FIG. 7D). The levels of phosphorylated ADAM17 and presenilin 2 were also decreased after EpAb2-6 or hEpAb2-6 treatment, while the MT201 antibody had no such effects (FIG. 7E). Our data suggested that EpEX and HGFR coordinately stimulate downstream HGFR signaling to promote tumor progression and cell invasion, so we wanted to further test the anti-tumor effects by simultaneously blocking both EpCAM and HGFR signaling. Furthermore, we found that HGFR inhibitor crizotinib could enhance the apoptotic effects of EpAb2-6 on HCT116 and HT29 cancer cells (FIG. 7F). In the cell invasion assay, crizotinib enhanced the inhibitory effect of EpAb2-6 on invasion in HCT116 and HT29 cells, as compared to control IgG (FIG. 7G).

### 2.7 EpAb2-6 Binds to the EGF-Like Domains I and II of EpCAM

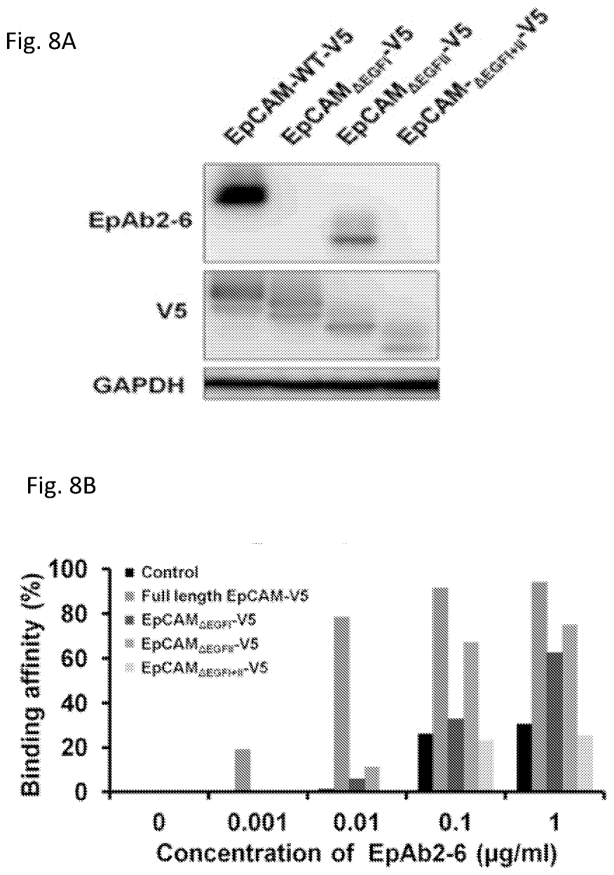

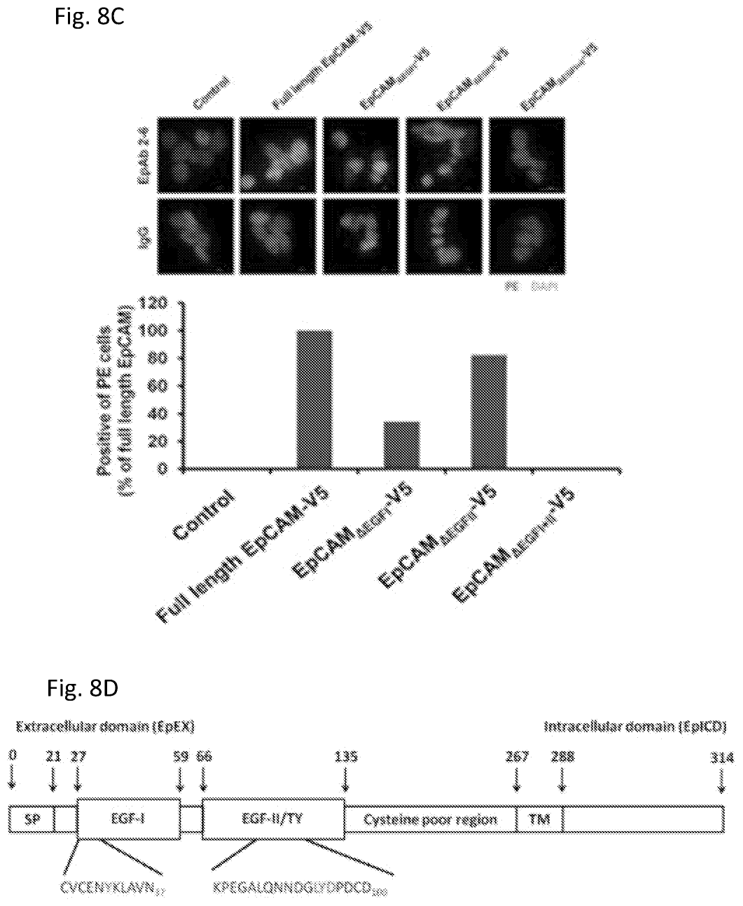

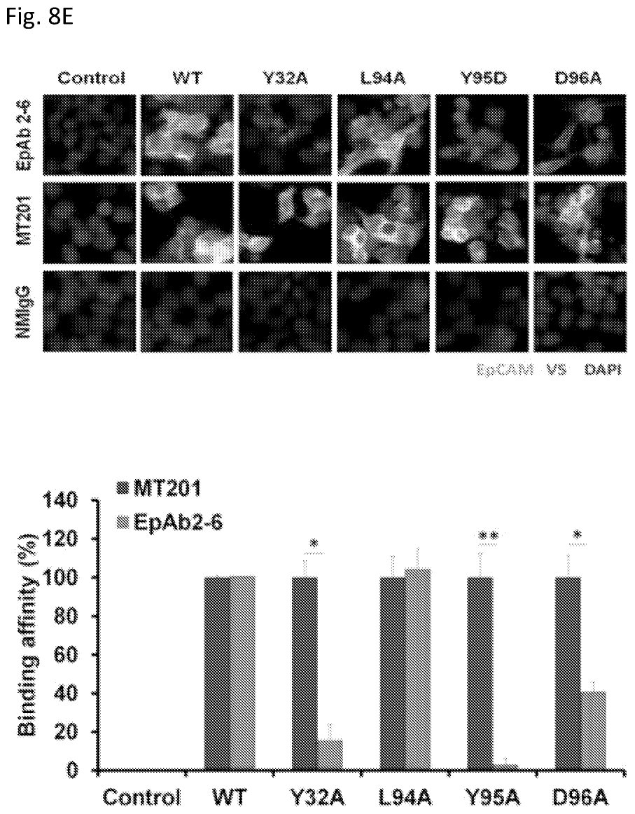

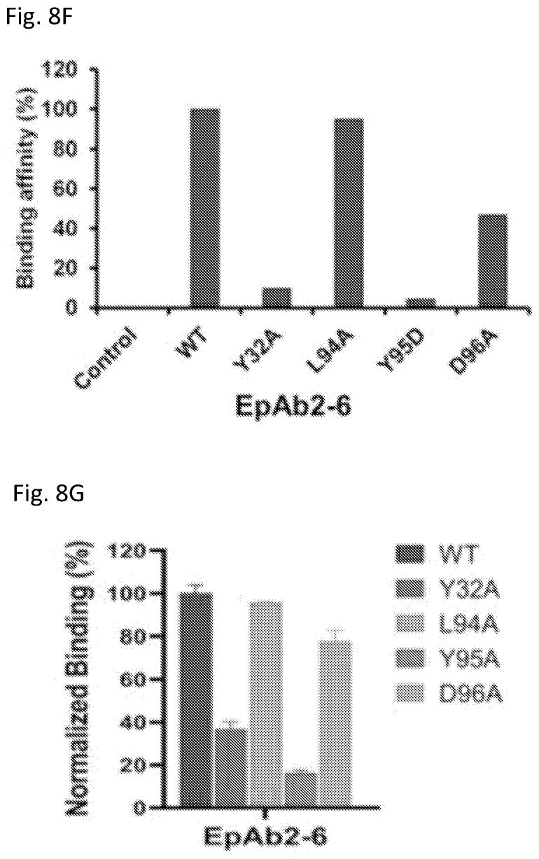

A previous study identified the binding epitope of EpAb2-6 antibody as the LYD motif in EpCAM, which corresponds to amino acid residues 94-96; in particular, residue 95 (Y95) plays a major role in EpAb2-6 binding (Liao et al., 2015). Here, we found that EpEX binds to HGFR through the EGF-like domains I and II (FIG. 1F and FIG. 1G), and EpAb2-6 can inhibit EpEX binding to HGFR (FIG. 6E and FIG. 6F). Therefore, we wanted to determine whether the antibody binds to EpCAM at both EGF-like domains of EpEX (FIG. 8A, FIG. 8B, FIG. 8C). To confirm that EpAb2-6 recognizes the LYD motif in EpCAM, we constructed cDNA sequences encoding the first (aa 27-59; EGF-I domain) and second (aa 66-135; EGF-II/TY domain) EGF-like repeats of EpCAM. PCR-based site-directed mutagenesis was then used to introduce mutations into each domain (FIG. 8D). The reactivity of EpAb2-6 antibody toward these EpCAM mutants was evaluated by immunofluorescence (FIG. 8E), flow cytometry (FIG. 8F), and cellular ELISA (FIG. 8G). Amino acid mutations at EpCAM positions Y32 (EGF-I domain) or Y95 (EGF-II domain) caused marked reductions in EpAb2-6 binding but did not affect MT201 binding. Thus, we conclude that EpAb2-6 binds to the EGF-I and EGF-II domains of EpEX, respectively targeting amino acid residues Y32 and Y95.

### 2.8 EpAb2-6 Improves the Efficacy of Crizotinib Therapy in Colon Cancer Animal Models

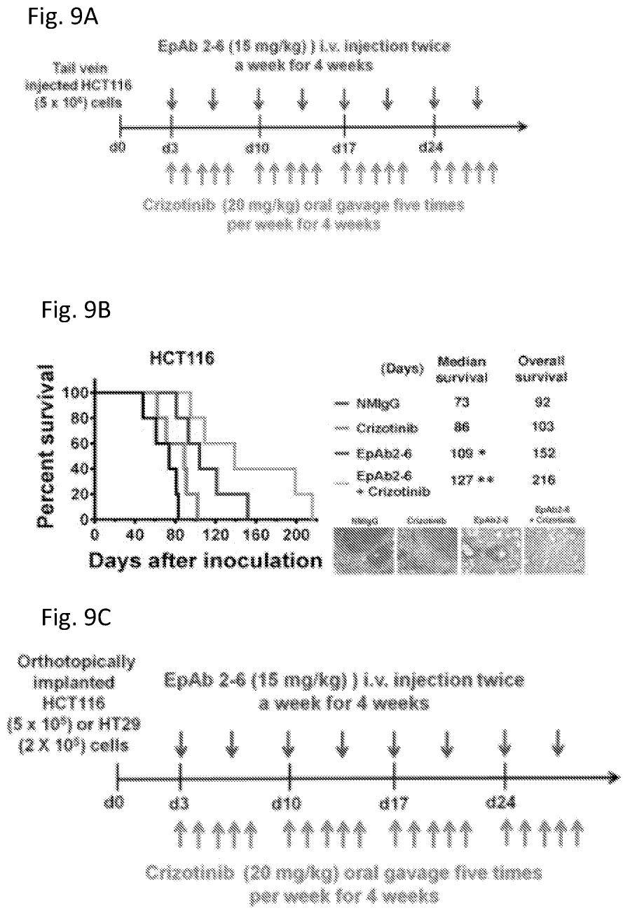

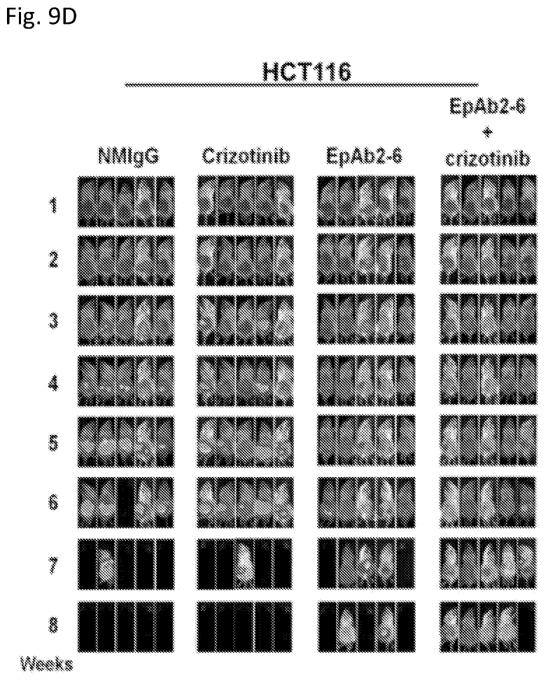

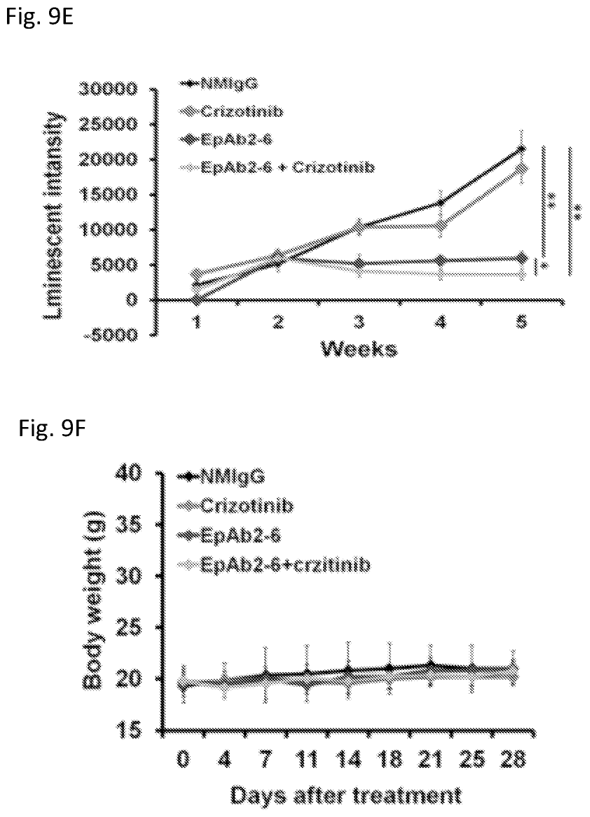

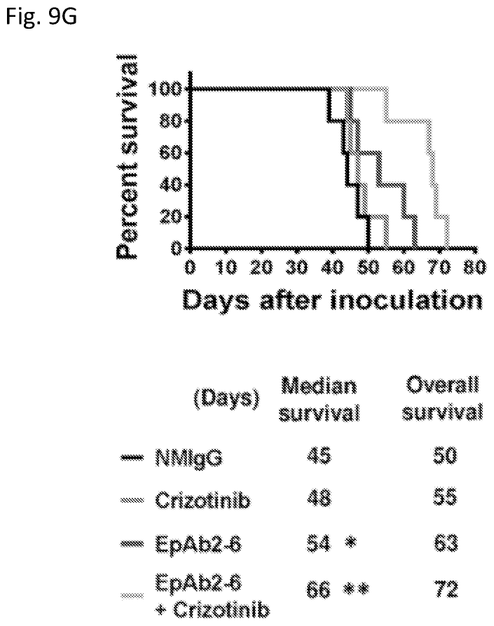

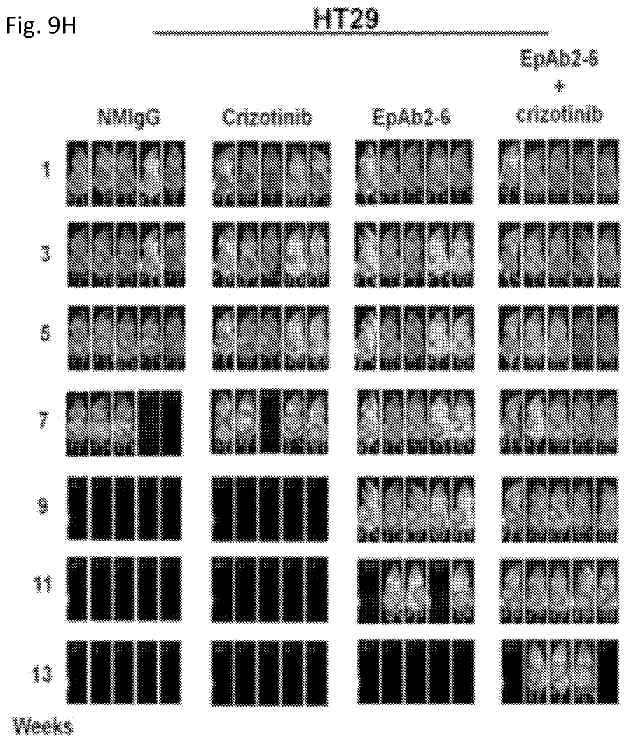

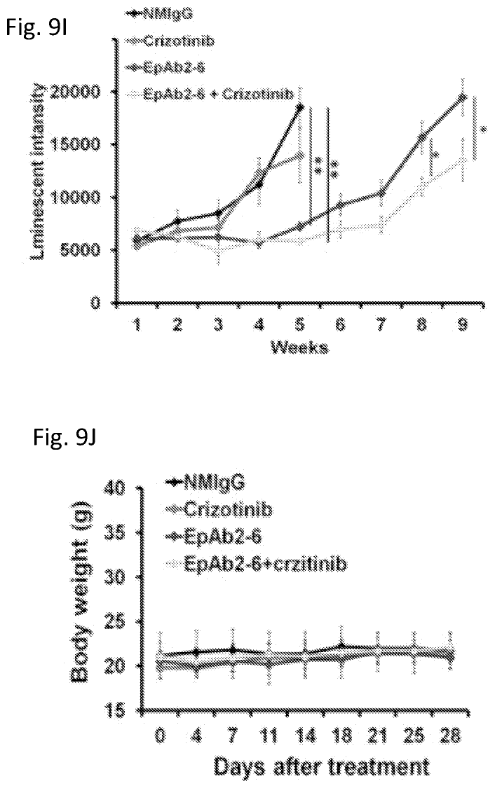

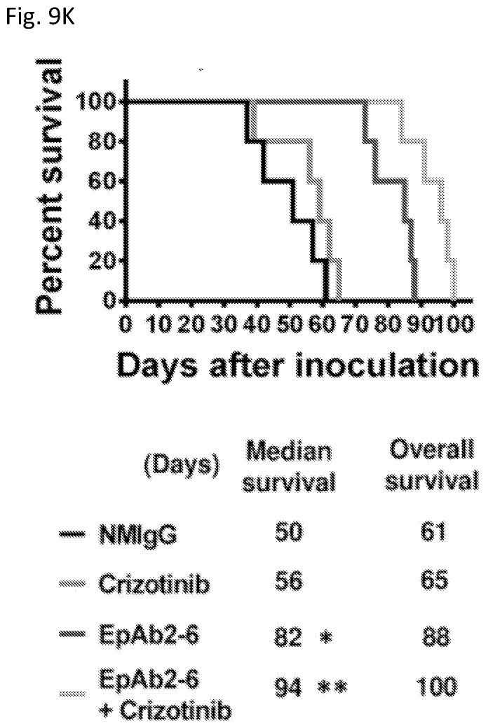

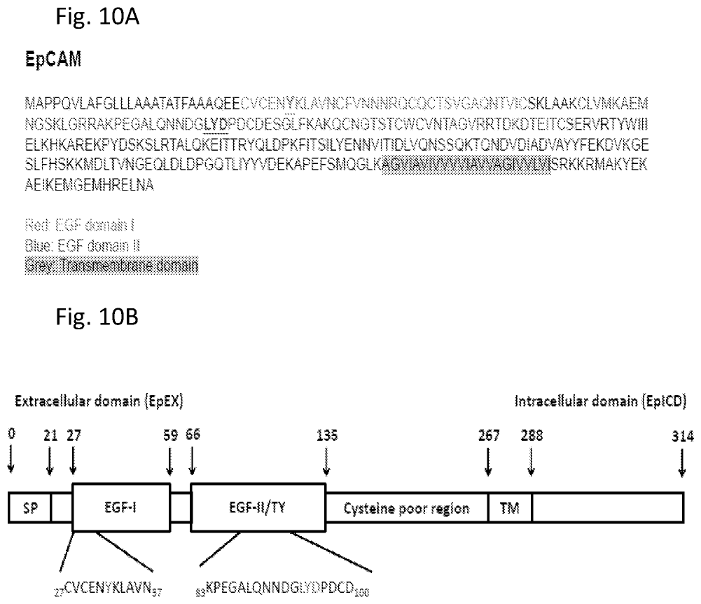

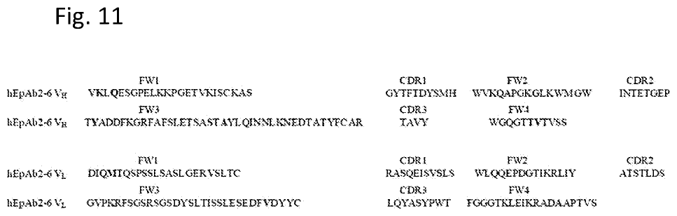

In the animal models, treatments were initiated 72 h post-transplantation (FIG. 9A). First, we examined the effect of crizotinib and EpAb2-6 on colon cancer cell HCT116 metastasis. NOD/SCID mice were injected intravenously with HCT116 cells and then co-treated with crizotinib and EpAb2-6, or an equivalent volume of control IgG, at 3 days after cell injection. The median and overall survival times of mice implanted with HCT116 cells receiving the combination of EpAb2-6 and crizotinib were increased compared to the control IgG group (FIG. 9B), supporting the idea that EpAb2-6 can improve the anti-metastatic action of crizotinib in vivo.

Next, we tested combined effects of EpAb2-6 and crizotinib as a therapeutic strategy in an orthotopic mouse model of colon cancer. As illustrated in FIG. 9C, tumor growth was assessed by in vivo monitoring of HT29-Luc and HCT116-Luc cells, which stably express firefly luciferase. Before initiation of the therapeutic treatment (3 days after tumor cell implantation), tumor growth could be observed in all mice. After treatment, bioluminescence intensities in mice receiving EpAb2-6 or the combination of EpAb2-6 and crizotinib were significantly decreased compared to the control IgG or crizotinib alone groups; similar effects were observed in orthotopic models transplanting HCT116 (FIG. 9D and FIG. 9E) or HT29 (FIG. 9H and FIG. 9I) cells. Moreover, the bodyweights were not significantly different between treatment groups in either transplanted with HCT116 (FIG. 9F) or HT29 (FIG. 9J) cells orthotopic model. The median and overall survival times of mice transplanted with HCT116 (FIG. 9G) or HT29 (FIG. 9K) cells receiving the combination of EpAb2-6 and crizotinib were significantly increased compared to the control IgG groups. Overall, the results of our experiments using metastatic and orthotopic animal models showed that all animals in the control IgG and crizotinib groups developed significant tumors and had poor survival. Meanwhile, the EpAb2-6-treated group had much slower tumor progression and showed higher median survival than the control IgG- or crizotinib-treated groups. Importantly, the attenuation of tumor progression was most pronounced in the combination treatment group.

### 3. Discussion

EpCAM expression is correlated with tumorigenesis and metastasis in many cancers, so we sought to elucidate the underlying mechanisms in this study. Here, we further show that EpEX-induced tumor progression and metastasis are mediated by HGFR signaling.

Many studies have made associations between high HGFR expression or activation and poor outcome in cancer patients (Birchmeier et al., 2003). High expression of HGFR is indicative of poor prognosis in thyroid carcinoma and non-small cell lung cancer (NSCLC), and it is a predictor of tumor invasion and lymph node metastases in colon cancer (Al-Saad et al, 2017; Takeuchi et al, 2003). Previous reports have also shown that in models of gastric cancer and CRC, blockade of HGFR signaling can reduce tumor cell growth and spread in vitro and in vivo (Smolen et al, 2006; Toiyama et al, 2012; Zou et al, 2007).

HGF is a cytokine that can modulate the proliferation of epithelial cells, and it is mainly expressed and secreted by mesenchymal cells (Lassus et al, 2006; Taher et al, 2002). The major coordinator of HGF signaling is HGFR, and the complex program induced by this signaling pathway promotes proliferation, survival, matrix degradation and migration. Together HGFR and HGF form the basis for an important epithelial and mesenchymal interaction that is necessary for wound closure and angiogenesis (Comoglio & Trusolino, 2002). Our results show that EpCAM knockout attenuates phosphorylation of HGFR in colon cancer cells, and the cell growth and migration capacities in EpCAM knockout HCT116 cells were significantly reduced compared to wild-type cells. When HGF treatment was restored in EpCAM knockout HCT16 cells, the EpCAM-induced effects on cell proliferation and migration were partially reversed. The ability of EpCAM to regulate HGFR phosphorylation suggests a strong possibility that this pathway may play an important role in colon cancer cells.

Tyrosine kinase inhibitors (TKIs) are small molecule drugs that can target activated RTKs regardless of ligand presence by preventing ATP from reaching the ATP-binding pocket of the kinase domain (Pasquini & Giaccone, 2018). Typically, drug resistance arises due to the acquisition of mutations in the RTK that can abolish the effect of the TKI, or by amplification of another RTK that can stimulate similar signaling, such as HGFR (Sacher et al, 2014). Previously our group used a Human Phospho-RTK Array Kit to screen for phosphorylation of RTKs in EpEX-Fc- and Fc-treated HCT116 colon cancer cells. The results showed that HGFR (MET)-tyrosine phosphorylation was stimulated by EpEX treatment (Liang et al., 2018). In this study, we found that incubation of HCT116 colon cancer cells with soluble EpEX-His protein induces HGFR phosphorylation. Interestingly, HGFR tyrosine kinase inhibitor (SU11274) could attenuate EpEX-mediated ERK and AKT phosphorylation. Furthermore, we confirmed that depletion of HGFR could attenuate EpEX-induced cell growth and invasion, consistent with the effects of the HGFR inhibitor.

Many studies have shown that EMT is associated with cancer progression and metastasis (Iwatsuki et al., 2010). The process of EMT involves a complex series of reversible events that can lead to the loss of epithelial cell adhesion and the induction of a mesenchymal phenotype in cells. EMT affords tumors with stem cell like plastic characteristics required for acquiring mesenchymal features, allowing tumor cells to disseminate and become more invasive. (Sacchetti et al, 2021; Thiery & Sleeman, 2006). Along with epithelial cell adhesion loss and induction of mesenchymal properties, cancer cells that undergo EMT also exhibit enhanced cell motility and invasion. Indicators of EMT include increased expression of mesenchymal markers, such as Vimentin, Snail and Slug, alongside decreased expression of epithelial markers like E-cadherin, which disrupts cell-celljunctions (Meng et al, 2012). Furthermore, cells that have undergone EMT also become resistant to apoptosis. Many reports have shown that EMT in different cancer types can promote resistance to various types of therapeutic drugs (Singh & Settleman, 2010). Blocking EMT for therapeutic purposes may be accomplished by targeting the components of the tumor microenvironment that contribute to activation of the EMT program in tumor cells (Shibue & Weinberg, 2017). For example, HGF induces the EMT program via HGFR signaling, thereby enhancing the invasive and metastatic potential of cancer cells by allowing the cells to survive in the blood stream in the absence of anchorage. Previous reports indicated FAK-PI3K/AKT and MAPK signaling pathways promoting migration and metastasis in colon cancer and glioblastoma (Golubovskaya, 2014; Song et al, 2016). Our data indicated that inhibitors of these molecules (i.e., SU11274, LY294002, U0126 and PF-562271) can attenuate EpEX-induced migration and invasion in HCT116 cells.

Inhibition of GSK3β by EpEX signaling can stabilize both β-catenin and Snail, which coordinately induce EMT-associated cell migration and invasion. Of note, EMT is correlated with high expression of non-phosphorylated (active) β-catenin and translocation of β-catenin into the nucleus, but the overexpression of β-catenin alone does not necessarily promote EMT-associated processes (Kim et al, 2000; Zhou et al., 2004). Additionally, Snail is a zinc-finger transcription factor that triggers EMT by repressing E-cadherin expression. Many oncogenic signals, such as PI3K/AKT, MAPK and Wnt, have been shown to inhibit GSK3β and thus cause the stabilization of Snail and subsequent EMT (Zhou et al., 2004). Our data showed that EpEX induces EMT and invasion by stabilizing active β-catenin and Snail via decreased GSK3β activity. Furthermore, our anti-EpCAM antibody inhibits EMT and invasion by increasing GSK3β activity, which leads to degradation of active β-catenin and Snail.

HGFR signaling is an important target for anticancer therapy, and substantial efforts have been made to develop antagonists of this pathway. Many small-molecule inhibitors of the HGFR tyrosine-kinase domain are currently being evaluated in clinical trials. HGFR overexpression is known to promote drug resistance in many cancer cells, resulting in poor treatment efficacy and shortened patient survival time (Yang et al, 2021; Zhao et al, 2020). For example, there is strong preclinical and clinical evidence showing that the HGFR signaling pathway is a key driver of multidrug resistance in multiple myeloma patients (Moschetta et al, 2013). The majority of HGFR inhibitors directly target its RTK activity rather than the HGF ligand because the main mechanism of HGFR activation is ligand-independent HGFR overexpression (Koch et al., 2020; Liang et al, 2020). Currently two non-selective HGFR TKIs are approved: crizotinib (first approved in 2011) for ALK- and ROS1-positive NSCLC, and cabozantinib (First approved in 2016) for thyroid cancer and kidney cancer (Kobayashi et al, 2016; Shaw et al, 2014). The relevance of HGFR inhibition is under intense evaluation, with several ongoing clinical trials on crizotinib. One crizotinib trial result suggested some promise for the treatment of NSCLC harboring HGFR exon 14-skipping mutations (Paik et al, 2015). The phase I MErCuRIC1 trial is evaluating the combination of crizotinib with a MEK inhibitor in a cohort of CRC patients harboring amplified HGFR and either wild-type or mutated RAS (NCT02510001) (Van Schaeybroeck et al, 2015). One previous report indicated crizotinib can block the HGF/STAT3/SOX13/HGFR feedback loop to suppress SOX13-mediated CRC migration, invasion and metastasis (Du et al, 2020).

Our group has produced an EpEX neutralizing antibody, EpAb2-6, which can be used to block the function of EpEX. EpAb2-6 treatment is known to disrupt signaling in the EpEX/EGFR/ADAM17 axis, which comprises a positive feedback loop to promote EpCAM cleavage and subsequently increase EpEX and EpICD production (Liang et al., 2018; Liao et al., 2015). Of note, decreased levels of phosphorylated HGFR were observed after EpAb2-6 treatment. Furthermore, EpAb2-6 could attenuate the invasion and migration capacity of HCT116 cells, but the capacity was partially restored after HGF treatment. Previous reports have demonstrated that EpAb2-6 can induce apoptosis, and we confirmed that after crizotinib treatment, HCT116 cells were sensitized to humanized-EpAb2-6- and EpAb2-6-induced apoptosis. Our results from the orthotopic colon cancer animal model also indicated that tumor growth was significantly inhibited after combined treatment of EpAb2-6 and crizotinib.

Among the potential treatments for NSCLC, crizotinib and other HGFR-targeting therapies have some of the most beneficial outcomes. This fact underscores the importance of deeply understanding the mechanisms that can be used to block HGFR activation. We found that EpCAM or EpEX can induce the HGFR-ERK-AKT signaling axis. According to these findings, EpCAM might be an excellent target for combination therapies with crizotinib. Indeed, in our experiments, EpAb2-6 decreased the level of phosphorylated HGFR and improved the therapeutic efficacy of crizotinib in animal models. Thus, our findings reveal a novel action of EpCAM in the regulation of HGFR signaling and suggest a new strategy of EpCAM/HGFR-targeted combination therapy.

In this study, we found that EpCAM/EpEX induces tumor progression and metastasis through ERK and FAK-AKT by inducing HGFR activation, GSK3β-Snail and β-catenin signaling in colon cancer cells. We further demonstrate that inhibition or depletion of EpCAM signaling leads to decreases in HGFR activation and its downstream signaling. Treatment with anti-EpCAM mAb EpAb2-6 reduced colon cancer progression and metastasis, and importantly, it improved the survival of mice in orthotopic tumor and metastasis models. Our data therefore suggest that therapeutic antibodies targeting EpCAM in combination with HGFR inhibitors may hold great potential for colon cancer therapy. The insights gained from these findings may be useful in the development of novel anticancer therapeutics that can inhibit metastasis and improve patient outcomes.

### REFERENCES

Al-Saad S, Richardsen E, Kilvaer T K, Donnem T, Andersen S, Khanehkenari M, Bremnes R M, Busund L-TJPo (2017) The impact of MET, IGF-1, IGF1R expression and EGFR mutations on survival of patients with non-small-cell lung cancer. 12: e0181527
An P, Chen F, Li Z, Ling Y, Peng Y, Zhang H, Li J, Chen Z, Wang HJO (2020) HDAC8 promotes the dissemination of breast cancer cells via AKT/GSK-30/Snail signals. 39: 4956-4969
Balzar M, Winter M J, de Boer C J, Litvinov SVJJomm (1999) The biology of the 17-1A antigen (Ep-CAM). 77: 699-712
Birchmeier C, Birchmeier W, Gherardi E, Woude GFVJNrMcb (2003) Met, metastasis, motility and more. 4: 915-925
Cao L, Wang F, Li S, Wang X, Huang D, Jiang RJCL (2019) PIM1 kinase promotes cell proliferation, metastasis and tumor growth of lung adenocarcinoma by potentiating the c-MET signaling pathway. 444: 116-126
Chang L, Graham P, Hao J, Ni J, Bucci J, Cozzi P, Kearsley J, Li YJCd, disease (2013) Acquisition of epithelial-mesenchymal transition and cancer stem cell phenotypes is associated with activation of the PI3K/Akt/mTOR pathway in prostate cancer radioresistance. 4: e875-e875
Chen H-N, Liang K-H, Lai J-K, Lan C-H, Liao M-Y, Hung S-H, Chuang Y-T, Chen K-C, Tsuei W W-F, Wu H-CJCR (2020) EpCAM signaling promotes tumor progression and protein stability of PD-L1 through the EGFR pathway. 80: 5035-5050
Comoglio P M, Giordano S, Trusolino LJNrDd (2008) Drug development of MET inhibitors: targeting oncogene addiction and expedience. 7: 504-516
Comoglio P M, Trusolino LJTJoci (2002) Series Introduction: Invasive growth: from development to metastasis. 109: 857-862
Dean M, Park M, Le Beau M M, Robins T S, Diaz M O, Rowley J D, Blair D G, Woude GFVJN (1985) The human met oncogene is related to the tyrosine kinase oncogenes. 318: 385-388
Dolle L, Theise N D, Schmelzer E, Boulter L, Gires 0, van Grunsven L A (2015) EpCAM and the biology of hepatic stem/progenitor cells. Am J Physiol-Gastr L 308: G233-G250
Du F, Li X, Feng W, Qiao C, Chen J, Jiang M, Qiu Z, Qian M, Tian D, Nie YJO (2020) SOX13 promotes colorectal cancer metastasis by transactivating SNAI2 and c-MET. 39: 3522-3540
Golubovskaya VMJFib (2014) Targeting FAK in human cancer: from finding to first clinical trials. 19: 687
Gumustekin M, Kargi A, Bulut G, Gozukizil A, Ulukus C, Oztop I, Atabey NJP, Research O (2012) HGF/c-Met overexpressions, but not met mutation, correlates with progression of non-small cell lung cancer. 18: 209-218
Hsu Y-T, Osmulski P, Wang Y, Huang Y-W, Liu L, Ruan J, Jin V X, Kirma N B, Gaczynska M E, Huang TH-MJCr (2016) EpCAM-regulated transcription exerts influences on nanomechanical properties of endometrial cancer cells that promote epithelial-to-mesenchymal transition. 76: 6171-6182
Iwatsuki M, Mimori K, Yokobori T, Ishi H, Beppu T, Nakamori S, Baba H, Mori MJCs (2010) Epithelial-mesenchymal transition in cancer development and its clinical significance. 101: 293-299
Jiao D, Wang J, Lu W, Tang X, Chen J, Mou H, Chen Q-yJMT-O (2016) Curcumin inhibited HGF-induced EMT and angiogenesis through regulating c-Met dependent PI3K/Akt/mTOR signaling pathways in lung cancer. 3: 16018
Jo M, Stolz D B, Esplen J E, Dorko K, Michalopoulos G K, Strom SCJJoBC (2000) Cross-talk between epidermal growth factor receptor and c-Met signal pathways in transformed cells. 275: 8806-8811
Kim K, Pang K M, Evans M, Hay EDJMbotc (2000) Overexpression of β-catenin induces apoptosis independent of its transactivation function with LEF-1 or the involvement of major G1 cell cycle regulators. 11: 3509-3523
Kim Y J, Choi J S, Seo J, Song J Y, Eun Lee S, Kwon M J, Kwon M J, Kundu J, Jung K, Oh EJIjoc (2014) MET is a potential target for use in combination therapy with EGFR inhibition in triple-negative/basal-like breast cancer. 134: 2424-2436
Kobayashi T, Fujimoto H, Gabazza ECJJotd (2016) Efficacy of crizotinib in ALK fusion variants. 8: E1381
Koch J P, Aebersold D M, Zimmer Y, Medovi MJO (2020) MET targeting: time for a rematch. 1-18
Lai A Z, Abella J V, Park MJTicb (2009) Crosstalk in Met receptor oncogenesis. 19: 542-551
Lassus P, Janer J, Haglund C, Karikoski R, Andersson L C, Andersson SJN (2006) Consistent expression of HGF and c-met in the perinatal lung. 90: 28-33
Li Z, Yanfang W, Li J, Jiang P, Peng T, Chen K, Zhao X, Zhang Y, Zhen P, Zhu JJCl (2018) Tumor-released exosomal circular RNA PDE8A promotes invasive growth via the miR-338/MACC1/MET pathway in pancreatic cancer. 432: 237-250
Liang H, Wang MJO, therapy (2020) MET oncogene in non-small cell lung cancer: mechanism of MET dysregulation and agents targeting the HGF/c-Met axis. 13: 2491
Liang K-H, Tso H-C, Hung S-H, Kuan I-I, Lai J-K, Ke F-Y, Chuang Y-T, Liu I-J, Wang Y-P, Chen R-HJCl (2018) Extracellular domain of EpCAM enhances tumor progression through EGFR signaling in colon cancer cells. 433: 165-175
Liao M-Y, Lai J-K, Kuo M Y-P, Lu R-M, Lin C-W, Cheng P-C, Liang K-H, Wu H-CJO (2015) An anti-EpCAM antibody EpAb2-6 for the treatment of colon cancer. 6: 24947
Lin C-W, Liao M-Y, Lin W-W, WangY-P, Lu T-Y, Wu H-CJJoBC (2012) Epithelial cell adhesion molecule regulates tumor initiation and tumorigenesis via activating reprogramming factors and epithelial-mesenchymal transition gene expression in colon cancer. 287: 39449-39459
Litvinov S V, van Driel W, van Rhijn C M, Bakker H, van Krieken H, Fleuren G J, Warnaar SOJTAjop (1996) Expression of Ep-CAM in cervical squamous epithelia correlates with an increased proliferation and the disappearance of markers for terminal differentiation. 148: 865
Mazzone M, Comoglio PMJTFJ (2006) The Met pathway: master switch and drug target in cancer progression. 20: 1611-1621
Meng F, Wu GJC, Reviews M (2012) The rejuvenated scenario of epithelial-mesenchymal transition (EMT) and cancer metastasis. 31: 455-467
Moschetta M, Basile A, Ferrucci A, Frassanito M A, Rao L, Ria R, Solimando A G, Giuliani N, Boccarelli A, Fumarola FJCcr (2013) Novel targeting of phospho-cMET overcomes drug resistance and induces antitumor activity in multiple myeloma. 19: 4371-4382
Mueller K L, Yang Z-Q, Haddad R, Ethier S P, Boemer JLJJoms (2010) EGFR/Met association regulates EGFR TKI resistance in breast cancer. 5: 8
Naldini L, Weidner K M, Vigna E, Gaudino G, Bardelli A, Ponzetto C, Narsimhan R P, Hartmann G, Zamegar R, Michalopoulos GKJTEj (1991) Scatter factor and hepatocyte growth factor are indistinguishable ligands for the MET receptor. 10: 2867-2878
Normanno N, De Luca A, Bianco C, Strizzi L, Mancino M, Maiello M R, Carotenuto A, De Feo G, Caponigro F, Salomon DSJG (2006) Epidermal growth factor receptor (EGFR) signaling in cancer. 366: 2-16
Ortiz-Zapater E, Lee R W, Owen W, Weitsman G, Fruhwirth G, Dunn R G, Neat M J, McCaughan F, Parker P, Ng TJPO (2017) MET-EGFR dimerization in lung adenocarcinoma is dependent on EGFR mtations and altered by MET kinase inhibition. 12: e0170798
Paik P K, Drilon A, Fan P-D, Yu H, Rekhtman N, Ginsberg M S, Borsu L, Schultz N, Berger M F, Rudin CMJCd (2015) Response to MET inhibitors in patients with stage I V lung adenocarcinomas harboring MET mutations causing exon 14 skipping. 5: 842-849
Pasquini G, Giaccone GJEOoID (2018)C-MET inhibitors for advanced non-small cell lung cancer. 27: 363-375
Peschard P, Park MJO (2007) From Tpr-Met to Met, tumorigenesis and tubes. 26: 1276-1285
Ponzetto C, Bardelli A, Zhen Z, Maina F, dalla Zonca P, Giordano S, Graziani A, Panayotou G, Comoglio PMJC (1994) A multifunctional docking site mediates signaling and transformation by the hepatocyte growth factor/scatter factor receptor family. 77: 261-271
Sacchetti A, Teeuwssen M, Verhagen M, Joosten R, Xu T, Stabile R, van der Steen B, Watson M M, Gusinac A, Kim WKJE (2021) Phenotypic plasticity underlies local invasion and distant metastasis in colon cancer. 10: e61461
Sacher A G, Janne P A, Oxnard GRJC (2014) Management of acquired resistance to epidermal growth factor receptor kinase inhibitors in patients with advanced non-small cell lung cancer. 120: 2289-2298
Schnell U, Kuipers J, Giepmans BNJBr (2013) EpCAM proteolysis: new fragments with distinct functions?33
Shattuck D L, Miller J K, Carraway K L, Sweeney CJCr (2008) Met receptor contributes to trastuzumab resistance of Her2-overexpressing breast cancer cells. 68: 1471-1477
Shaw A T, Ou S-H I, Bang Y-J, Camidge D R, Solomon B J, Salgia R, Riely G J, Varella-Garcia M, Shapiro G I, Costa DBJNEJoM (2014) Crizotinib in ROS1-rearranged non-small-cell lung cancer. 371: 1963-1971
Shibue T, Weinberg RAJNrCo (2017) EMT, CSCs, and drug resistance: the mechanistic link and clinical implications. 14: 611
Simiczyjew A, Dratkiewicz E, Van Troys M, Ampe C, Styczen I, Nowak DJC (2018) Combination of EGFR inhibitor lapatinib and MET inhibitor foretinib inhibits migration of triple negative breast cancer cell lines. 10: 335
Singh A, Settleman JJO (2010) EMT, cancer stem cells and drug resistance: an emerging axis of evil in the war on cancer. 29: 4741-4751
Smolen G A, Sordella R, Muir B, Mohapatra G, Barmettler A, Archibald H, Kim W J, Okimoto R A, Bell D W, Sgroi DCJPotNAoS (2006) Amplification of MET may identify a subset of cancers with extreme sensitivity to the selective tyrosine kinase inhibitor PHA-665752. 103: 2316-2321
Song G, Xu S, Zhang H, Wang Y, Xiao C, Jiang T, Wu L, Zhang T, Sun X, Zhong LJJoE et al (2016) TIMP1 is a prognostic marker for the progression and metastasis of colon cancer through FAK-PI3K/AKT and MAPK pathway. 35: 1-12
Sun Y, Liu W-Z, Liu T, Feng X, Yang N, Zhou H-FJJoR, Transduction S (2015) Signaling pathway of MAPK/ERK in cell proliferation, differentiation, migration, senescence and apoptosis. 35: 600-604
Taher T E, Tjin E P, Beuling E A, Borst J, Spaargaren M, Pals STJTJoI (2002) c-Cbl is involved in Met signaling in B cells and mediates hepatocyte growth factor-induced receptor ubiquitination. 169: 3793-3800
Takeuchi H, Bilchik A, Saha S, Turner R, Wiese D, Tanaka M, Kuo C, Wang H-J, Hoon DSJCCR (2003) c-MET expression level in primary colon cancer: a predictor of tumor invasion and lymph node metastases. 9: 1480-1488
Tang Z, Du R, Jiang S, Wu C, Barkauskas D, Richey J, Molter J, Lam M, Flask C, Gerson SJBjoc (2008) Dual MET-EGFR combinatorial inhibition against T790M-EGFR-mediated erlotinib-resistant lung cancer. 99: 911-922
Thiery J P, Sleeman JPJNrMcb (2006) Complex networks orchestrate epithelial-mesenchymal transitions. 7: 131-142
Toiyama Y, Yasuda H, Saigusa S, Matushita K, Fujikawa H, Tanaka K, Mohri Y, Inoue Y, Goel A, Kusunoki MJIjoc (2012) Co-expression of hepatocyte growth factor and c-Met predicts peritoneal dissemination established by autocrine hepatocyte growth factor/c-Met signaling in gastric cancer. 130: 2912-2921
Van Schaeybroeck S, Rolfo C D, Elez E, Kelly S, Houlden J, Collins L, Love S, Andre T, Lawler M, Di Nicolantonio F, 2015. MErCuRIC1: A Phase I study of MEK1/2 inhibitor PD-0325901 with cMET inhibitor crizotinib in RAS M T and RAS W T (with aberrant c-MET) metastatic colorectal cancer (mCRC) patients. American Society of Clinical Oncology.
Yang Yn, Wang C, Dai C, Liu X, Li W, Huang M, Zhao X, Ji D, Li J, Guo WJABeBS (2021) Amplification and expression of c-MET correlate with poor prognosis of patients with gastric cancer and upregulate the expression of PDL1. 53: 547-557
Zhao C, Guo R, Guan F, Ma S, Li M, Wu J, Liu X, Li H, Yang BJSr (2020) MicroRNA-128-3p Enhances the Chemosensitivity of Temozolomide in Glioblastoma by Targeting c-Met and EMT. 10: 1-12
Zhou B P, Deng J, Xia W, Xu J, Li Y M, Gunduz M, Hung M-CJNcb (2004) Dual regulation of Snail by GSK-3β-mediated phosphorylation in control of epithelial-mesenchymal transition. 6: 931-940
Zou H Y, Li Q, Lee J H, Arango M E, McDonnell S R, Yamazaki S, Koudriakova T B, Alton G, Cui J J, Kung P-PJCr (2007) An orally available small-molecule inhibitor of c-Met, PF-2341066, exhibits cytoreductive antitumor efficacy through antiproliferative and antiangiogenic mechanisms. 67: 4408-4417

1. A method for treating cancer, comprising
administering to a subject in need thereof
(i) an effective amount of a first inhibitory agent that inhibits the activation of epithelial cell adhesion molecule (EpCAM) signaling; and
(ii) an effective amount of a second inhibitory agent that inhibits the activation of HGFR signaling.

2. The method of claim 1, wherein the first inhibitory agent reduces production (or release) of an extracellular domain (EpEX) of EpCAM, blocks binding of EpEX to HGFR, and/or inhibits EpEX-induced HGFR phosphorylation.

3. The method of claim 1, wherein the second inhibitory agent blocks binding of HGF to HGFR.

4. The method of claim 1, wherein the first inhibitory agent is an antibody directed to EpEX or an antigen-binding fragment thereof.

5. The method of claim 4, wherein the antibody specifically binds to epidermal growth factor (EGF)-like domains I and I I.

6. The method of claim 4, wherein the antibody has a specific binding affinity to an epitope within the sequence of CVCENYKLAVN (aa 27 to 37) (SEQ I D N O: 20) located in the EGF-like domain I, and KPEGALQNNDGLYDPDCD (aa 83 to 100) (SEQ I D N O: 19) located in the EGF-like domain I I.

7. The method of claim 4, wherein the antibody or antigen-binding fragment comprises
(a) a heavy chain variable region (V H) which comprises a heavy chain complementary determining region 1 (H C CDR1) comprising the amino acid sequence of SEQ I D N O: 2, a heavy chain complementary determining region 2 (H C CDR2) comprising the amino acid sequence of SEQ I D N O: 4, and a heavy chain complementary determining region 3 (H C CDR3) comprising the amino acid sequence of SEQ I D N O: 6; and
(b) a light chain variable region (V L) which comprises a light chain complementary determining region 1 (L C CDR1) comprising the amino acid sequence of SEQ I D N O: 9, a light chain complementary determining region (L C CDR2) comprising the amino acid sequence of SEQ I D N O: 11, and a light chain complementary determining region 3 (L C CDR3) comprising the amino acid sequence of SEQ I D N O: 13.

8. The method of claim 1, wherein the first inhibitory agent is effective in inhibiting phosphorylation of TACE and PS2 signaling.

9. The method of claim 1, wherein the second inhibitory agent is selected from the group consisting of foretinib, crizotinib and cabozantinib.

10. The method of claim 1, wherein the method is effective in inducing apoptosis of cancer cells.

11. The method of claim 1, wherein the method is effective in inhibiting migration/invasion of cancer cells and/or reducing tumor size.

12. The method of claim 1, wherein the method is effective in prolonging survival of the subject.

13. The method of claim 1, wherein the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer.

14. A kit or a pharmaceutical composition comprising:
(i) a first inhibitory agent that inhibits the activation of EpCAM signaling; and
(ii) a second inhibitory agent that inhibits the activation of HGFR signaling.

15. The kit or pharmaceutical composition of claim 14, wherein
the first inhibitory agent reduces production (or release) of an extracellular domain (EpEX) of EpCAM, blocks binding of EpEX to HGFR, and/or inhibits EpEX-induced HGFR phosphorylation, and
the second inhibitory agent blocks binding of HGF to HGFR.

16-21. (canceled)


SEQUENCE LISTING (35 sequences)

SEQ ID NO: 1 - protein, 24 aa, Homo sapiens
VKLQESGPELKKPGETVKISCKAS

SEQ ID NO: 2 - protein, 10 aa, Homo sapiens
GYTFTDYSMH

SEQ ID NO: 3 - protein, 15 aa, Homo sapiens
WVKQAPGKGLKWMGW

SEQ ID NO: 4 - protein, 8 aa, Homo sapiens
INTETGEP

SEQ ID NO: 5 - protein, 40 aa, Homo sapiens
TYADDFKGRFAFSLETSASTAYLQINNLKNEDTATYFCAR

SEQ ID NO: 6 - protein, 4 aa, Homo sapiens
TAVY

SEQ ID NO: 7 - protein, 11 aa, Homo sapiens
WGQGTTVTVSS

SEQ ID NO: 8 - protein, 23 aa, Homo sapiens
DIQMTQSPSSLSASLGERVSLTC

SEQ ID NO: 9 - protein, 11 aa, Homo sapiens
RASQEISVSLS

SEQ ID NO: 10 - protein, 15 aa, Homo sapiens
WLQQEPDGTIKRLIY

SEQ ID NO: 11 - protein, 7 aa, Homo sapiens
ATSTLDS

SEQ ID NO: 12 - protein, 32 aa, Homo sapiens
GVPKRFSGSRSGSDYSLTISSLESEDFVDYYC

SEQ ID NO: 13 - protein, 9 aa, Homo sapiens
LQYASYPWT

SEQ ID NO: 14 - protein, 19 aa, Homo sapiens
FGGGTKLEIKRADAAPTVS

SEQ ID NO: 15 - protein, 112 aa, Homo sapiens
VKLQESGPELKKPGETVKISCKASGYTFTDYSMHWVKQAPGKGLKWMGWINTETGEPTYADDFKGRFAFSLETSASTAYLQINNLKNEDTATYFCARTAVYWGQGTTVTVSS

SEQ ID NO: 16 - protein, 116 aa, Homo sapiens
DIQMTQSPSSLSASLGERVSLTCRASQEISVSLSWLQQEPDGTIKRLIYATSTLDSGVPKRFSGSRSGSDYSLTISSLESEDFVDYYCLQYASYPWTFGGGTKLEIKRADAAPTVS

SEQ ID NO: 17 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 18 - protein, 9 aa, Homo sapiens
VGAQNTVIC

SEQ ID NO: 19 - protein, 19 aa, Homo sapiens
KPEGALQNNDGLYDPDCDE

SEQ ID NO: 20 - protein, 11 aa, Homo sapiens
CVCENYKLAVN

SEQ ID NO: 21 - DNA, 20 bp, Artificial Sequence
  EpCAM gRNA
gtgcaccaactgaagtacac

SEQ ID NO: 22 - DNA, 30 bp, Artificial Sequence
  1st forward mutagenic deletion primer
gcagctcaggaagaatcaaagctggctgcc

SEQ ID NO: 23 - DNA, 30 bp, Artificial Sequence
  1st reverse mutagenic deletion primer
ggcagccagctttgattcttcctgagctgc

SEQ ID NO: 24 - DNA, 30 bp, Artificial Sequence
  2nd forward primer
aagctggctgccaaatctgagcgagtgaga

SEQ ID NO: 25 - DNA, 30 bp, Artificial Sequence
  2nd reverse primer
tctcactcgctcagatttggcagccagctt

SEQ ID NO: 26 - DNA, 18 bp, Artificial Sequence
  EPCAM primer F
ctccacgtgctggtgtgt

SEQ ID NO: 27 - DNA, 26 bp, Artificial Sequence
  EPCAM primer R
tgttttagttcaatgatgatccagta

SEQ ID NO: 28 - DNA, 22 bp, Artificial Sequence
  E-cadherin primer F
ggaactatgaaaagtgggcttg

SEQ ID NO: 29 - DNA, 20 bp, Artificial Sequence
  E-cadherin primer R
aaattgccaggctcaatgac

SEQ ID NO: 30 - DNA, 20 bp, Artificial Sequence
  VIM primer F
gtttcccctaaaccgctagg

SEQ ID NO: 31 - DNA, 18 bp, Artificial Sequence
  VIM primer R
agcgagagtggcagagga

SEQ ID NO: 32 - DNA, 19 bp, Artificial Sequence
  SNAIL primer F
cttcggctccaggagagtc

SEQ ID NO: 33 - DNA, 21 bp, Artificial Sequence
  SNAIL primer R
ttcccactgtcctcatctgac

SEQ ID NO: 34 - DNA, 21 bp, Artificial Sequence
  GAPDH primner F
cttcaccaccatggaggaggc

SEQ ID NO: 35 - DNA, 21 bp, Artificial Sequence
  GAPDH primer R
ggcatggactgtggtcatgag